(19) United States
(12) Patent Application Publication
(10) Pub. No.: US 2004/0214190 A1
(43) Pub. Date: Oct. 28, 2004

(54) Mammalian c-type lectins

(76) Inventors: Eric A Butz, Seattle, WA (US); Dirk M Anderson, Seattle, WA (US)

Correspondence Address: IMMUNEX CORPORATION, LAW DEPARTMENT, 1201 AMGEN COURT WEST, SEATTLE, WA, 98119, US

(21) Appl. No.: 10/492,100

(22) PCT Filed: Oct. 4, 2002
(86) PCT No.: PCT/US02/31996

Related U.S. Application Data
(60) Provisional application No. 60/328,026, filed on Oct. 9, 2001.

(51) Int. Cl. C12Q 1/68; C07H 21/04; C12N 5/06; C07K 14/47

(52) U.S. Cl. 435/6; 435/69.1; 435/320.1; 435/325; 530/396; 536/23.5

(57) ABSTRACT

The present invention provides novel mammalian C-type lectin polypeptides associated with antigen presenting cells. Designated as Dendritic Cell C-type Lectins (DCL), the following four novel genes have been discovered: DCL 1, DCL 2, DCL 3 and DCL 4, as well as splice variants svDCL 2, svDCL 3 and svDCL 4, and a human homologue herein designated DCL 5. The present invention provides polynucleotides encoding DCL polypeptides, recombinant expression vectors, host cells transfected with the recombinant expression vectors, methods of producing and isolating the inventive polypeptides and various screening assays. Therapeutic compositions and methods of treating various diseases are also provided.

### CROSS-REFERENCE TO RELATED APPLICATIONS

This application claims the benefit under 35 U.S.C. §119(e) to U.S. Provisional Patent Application Serial No. 60/328,026, filed Oct. 9, 2001, the disclosure of which is incorporated herein by reference.

### FIELD OF THE INVENTION

The present invention provides novel C-type lectin family members expressed in antigen presenting cells, and in one particular embodiment, C-type lectins upregulated on dendritic cells in response to stimulation with bacterial lipopolysaccharide (LPS).

### BACKGROUND OF THE INVENTION

Host defense systems rely on innate and adaptive immunity to protect the host from infectious agents and injury. The innate immune system includes several immunoregulatory components such as complement, natural killer cells and phagocytic cells and is characterized by the capacity to rapidly recognize pathogenic and/or tissue injury as well as the ability to send a variety of signals to cells of the adaptive immune system. Cells of the innate system use a variety of receptors to recognize patterns shared between pathogens, for example, bacterial lipopolysaccharide (LPS), carbohydrates, and double-stranded viral RNA. The adaptive immune system, or humoral and cell mediated immunity, is characterized by the ability to rearrange genes of the immunolglobulin family, permitting a large diversity of antigen-specific clones and immunological memory. Antigen presenting cells (APCs) serve to instruct and regulate the cells of the adaptive immune system.

Dendritic cells (DCs) are unique APCs in that they are the only cells known to induce primary T-cell responses, thereby allowing antigen-specific immune responses and establishing immunological memory. DCs are professional APCs that are especially efficient stimulators of B and T lymphocytes. DCs have the capacity to prime naïve T cells to mismatched MHC, superantigens (Bhardwaj, N., et al., J. Exp. Med. 178, 633-642 (1993)), proteins from infectious agents (Inaba, K., et al., J. Exp. Med., 178, 479-488 (1993)) and tumors (Mayordomo, J. I., et al., Nature Med., 1, 1297-1302, (1995); Hsu, F. J., et al., Nature Med., 2, 52-58, (1996)). DCs are extremely efficient in activating T-cells, and in mixed lymphocyte reactions, one DC may activate from 100 to 3,000 T-cells. Researchers have yet to pinpoint the basis for the T-cell binding and activation efficiency of DCs, but it appears that the unique stimulatory properties of DCs may be attributable in part to the fact that MHC products and MHC-peptide complexes are 10 to 100 times higher on DCs than on other APCs, such as B-cells and monocytes (Inaba, K., et al., J. Exp. Med., 186, 665-672 (1997)). In addition, subsets of mature DCs resist the suppressive effects of IL-10 and synthesize high levels of IL 12, which in turn enhances innate immunity in the form of natural killer cells and acquired immunity by T and B cells (Koch, F., et al., J. Exp. Med., 184, 741-747 (1936)). Furthermore, DCs upregulate and express many accessory molecules that interact with receptors on T cells to enhance adhesion and costimulation, such as LFA-3/CD58, ICAM-1/CD54 and B7-2/CD86 (Banchereau, et al., Nature, 392, 245-252, (1998)).

DCs are located in most tissues where they serve a sentinel role by capturing and processing antigens. In one form, DC precursors migrate from bone marrow and circulate in the blood to specific sites in the body, where they mature. This trafficking is partially directed by expression of chemokine receptors and adhesion molecules. This link between DC traffic pattern and function has led to the investigation of the chemokine responsiveness of DC during their development and maturation. For a review of the effect of chemokines on dendritic cell subsets, see Dieu-Nosjean, J. Leuk. Biol. 66(2):252-62, 1999. In general, upon exposure to antigen and activation signals the DCs are activated and upregulate costimulatory and adhesion molecules, and leave tissues to migrate via the afferent lymphatics to the T-cell rich paracortex of the draining lymph nodes. The activated DCs then secrete chemokines and cytokines involved in T-cell homing and activation, and present processed antigen to T-cells. For example, DC-SIGN, a DC-specific C-type lectin, has been shown to support tethering and rolling of DC-SIGN-positive cells on the vascular ligand ICAM-2. This process may be a prerequisite for emigration from the blood, and it has been shown that the DC-SIGN:ICAM-2 interaction regulates chemokine-induced transmigration of DCs across both resting and activated endothelium (Teunis, B. H., et al., Nature 1:353-357, 2000). Furthermore, DC-SIGN has been demonstrated to mediate transient adhesion with ICAM-3 expressed on resting T-cells and that binding to ICAM-3 plays an important role in establishing the first contact between DC and T cells and facilitates subsequent low-avidity interactions with other adhesion molecules that enable productive T cell receptor engagement followed by adhesion strengthening (Teunis, B. H., et al., Cell 100:575-585, 2000).

Immature DCs are very efficient in antigen capture and use several pathways, such as macropinocytosis; receptor-mediated endocytosis via C-type lectin receptors or Fcγ receptor types I (CD64) and II (CD32) for internalization of immune complexes and phagocytosis of particulates. Phagocytosis of particulates include apoptotic and necrotic cell fragments involving CD36 and αVβ3 or αVβ5 integrins (Albert, M L., et al., J. Exp. Med. 188:1359-68, 1998; Rubartelli, A., et al., Eur. J. Immunol. 27:1893-900, 1997), viruses and bacteria, as well as intracellular parasites (Moll, H., et al., Immunol Today, 14:383-87, 1993). DCs also have the capacity to internalize peptide-loaded heat shock proteins gp96 and Hsp70 (Arnold-Schild, D., et al., J. Immunol. 162:3757-60, 1999). CD91, the widely expressed α2-macroglobulin receptor, has been shown to be a receptor for gp96 (Binder, J. R., et al., J. Immunol. 1:151-55, 2000). In certain DC subtypes, the uptake of antigen induces the immature DC to undergo phenotypic and functional changes that culminate in the complete transition from an antigen capturing cell to an antigen presenting cell.

The role of receptor-mediated endocytosis via C-type (Ca²⁺-dependent) lectin receptors, such as the mannose receptor (Hart, D. N., et al., Blood 90:3245:87, 1997) and DEC-205 (Jiang, W., et al., Nature 375:151-55, 1995), is a subject of great interest in DC biology. The mannose-binding-lectin pathway (MBL), which includes the complement activation pathway, is mediated by the binding of the MBL to carbohydrates via a carbohydrate recognition domain (CRD). The CRD binding is sugar-selective and calcium dependent. The MBL binds to an array of carbohydrate structures on the surfaces of microorganisms, which in turn, mediates an antimicrobial response by direct killing via complement through the lytic membrane attack complex or by promoting phagocytosis of the organism. The capacity to discriminate between self and non-self structures resides in the specificity of the CRD and in the spatial arrangement of the CRDs. For a review of the MBL pathway, see Gadjeva, M., et al., Curr. Opin. Immunol. 13:74-78, 2001.

A number of groups have identified several new C-type lectins unique to macrophages and DC, such as the murine macrophage-restricted C-type lectin (mMCL) (Balch, S., et al., J. Biol. Chem. 273:18656-64, 1998); Langerin, the Langerhans cell-specific C-type lectin (Valladeau, J., Immunity 12:71-81, 2000), Mincle, a macrophage-inducible C-type lectin that is a transcriptional target of NF-IL6 in murine peritoneal macrophages (Matsumoto, M., et al., J. Immunol. 163:503948, 1999); DCIR, the human dendritic cell immumoreceptor, a type II glycoprotein with homology to the macrophage lectin and hepatic asialoglycoprotein receptors (Bates, E., et al., J. Immunol. 163:1973-83, 1999 and U.S. Pat. No. 6,277,959); and, murine Dectin-1 and Dectin-2 (DC-associated C-type lectins; Ariizumi, K., et al., J. Biol. Chem., 275:20157-167, 2000 and Ariizumi, K., et al., J. Biol. Chem., 275:11957-963, 2000, respectively), which are thought to be involved in delivering T-cell costimulatory signals.

To date, the role of C-type lectins in APC and particularly DC biology is not fully understood. For example, C-type lectins may play a role in APC/DC activation, differentiation, maturation, migration, antigen capture, antigen processing and presentation, as well as interactions with T, B and other cells of the immune system. Manipulation of these aspects of APC/DC biology may be useful in the areas of inflammation, oncology, autoimmunity, infectious disease, transplantation, adjuvants and vaccines. The present invention addresses such issues.

### SUMMARY OF THE INVENTION

The present invention is based upon the discovery of novel C-type lectin family members expressed in APCs, and in one particular embodiment, C-type lectins upregulated on DCs in response to stimulation with LPS.

In another aspect, the present invention provides novel mammalian C-type lectin polypeptides associated with murine dendritic cells herein designated Dendritic Cell C-type Lectins (DCL). Namely, DCL 1 (SEQ ID NO:2), DCL 2 (SEQ ID NO:6), DCL 3 (SEQ ID NO:12) and DCL 4 (SEQ ID NO:22), as well as splice variants svDCL 2 (SEQ ID NO:10), svDCL 3 (SEQ ID NO:16) and svDCL 4 (SEQ ID NO:22), and a human homologue, herein designated DCL 5 (SEQ ID NO:24). The present invention provides polynucleotides encoding DCL polypeptides and recombinant expression vectors that include polynucleotides encoding DCL polypeptides. The present invention additionally provides methods for isolating DCL polypeptides and methods for producing recombinant DCL polypeptides by cultivating host cells transfected with the recombinant expression vectors under conditions appropriate for expressing C-type lectin polypeptides and recovering the expressed lectin polypeptides

The invention provides an isolated polypeptide consisting of, consisting essentially of, or more preferably, comprising an amino acid sequence selected from the group consisting of:

(a) the amino acid sequence of SEQ ID NO:2, 6, 10, 12, 16, 18, 22 or 24;

(b) an amino acid sequence selected from the group consisting of: amino acids 1 through 245 of SEQ ID NO:2 and amino acids 77 through 245 of SEQ ID NO:2;

(c) the amino acid sequence of SEQ ID NO:2 comprising all or part of the extracellular domain having at least one DCL activity;

(d) fragments of the amino acid sequences of any of (a)-(c) comprising at least 25 contiguous amino acids having at least one DCL activity;

(e) fragments of the amino acid sequences of any of (a)-(c) comprising at least 25 contiguous amino acids having a C-type lectin domain;

(f) fragments of the amino acid sequences of any of (a)-(c) comprising at least 25 contiguous amino acids having an immunoreceptor tyrosine-based inhibitory-like motif (ITIM) amino acid sequences;

(g) fragments of the amino acid sequences of any of (a)-(c) comprising at least 25 contiguous amino acids that are immunogenic;

(h) amino acid sequences comprising at least 25 amino acids and sharing amino acid identity with the amino acid sequences of any of (a)-(g), wherein the percent amino acid identity is selected from the group consisting of: at least 80%, at least 85%, at least 90%, at least 95%, at least 97.5%, at least 99%; and at least 99.5%, wherein the amino acid sequences have at least one DCL activity; and

(i) amino acid sequences comprising at least 20 amino acids having at least one modification selected from the group consisting of amino acid substitutions, amino acid insertions, amino acid deletions, C-terminal truncation, and N-terminal truncation, wherein the amino acid sequences have at least one DCL activity.

Other aspects of the invention are isolated nucleic acids encoding polypeptides of the invention, with a preferred embodiment being an isolated nucleic acid consisting of, or more preferably, comprising a nucleotide sequence selected from the group consisting of:

(a) SEQ ID NO:1, 5, 9, 11, 15, 17; 21 and 23; and

(b) allelic variants of (a).

The invention also provides an isolated genomic nucleic acid corresponding to the nucleic acids of the invention.

Other aspects of the invention are isolated nucleic acids encoding polypeptides of the invention, and isolated nucleic acids, preferably having a length of at least 15 contiguous nucleotides, that hybridize under conditions of moderate stringency to the nucleic acids encoding polypeptides of the invention. In preferred embodiments of the invention, such nucleic acids encode a polypeptide having C-type lectin polypeptide activity, or comprise a nucleotide sequence that shares nucleotide sequence identity with the nucleotide sequences of the nucleic acids of the invention, wherein the percent nucleotide sequence identity is selected from the group consisting of: at least 80%, at least 85%, at least 90%, at least 95%, at least 97.5%, at least 99%, and at least 99.5%.

Further provided by the invention are expression vectors and recombinant host cells comprising at least one nucleic acid of the invention, and preferred recombinant host cells wherein said nucleic acid is integrated into the host cell genome.

Also provided is a process for producing a polypeptide encoded by the nucleic acids of the invention, comprising culturing a recombinant host cell under conditions promoting expression of said polypeptide, wherein the recombinant host cell comprises at least one nucleic acid of the invention. In another aspect of the invention, the polypeptide produced by said process is provided.

Further within the scope of the present invention are processes for purifying or separating DCL polypeptides or cells that express DCL polypeptides. Such processes include binding at least one binding partner to a solid phase matrix and contacting a mixture containing a DCL polypeptide(s) to which the DCL polypeptide(s) binds, or a mixture of cells expressing the DCL polypeptide(s), and then separating the contacting surface and the solution.

Further aspects of the invention include isolated antibodies, monoclonal antibodies, human or humanized antibodies and the like, as described in more detail below, that bind to the polypeptides of the invention. Further embodimets include such antibodies that agonize the DCL activity of said polypeptides. Further embodiments include such antibodies that inhibit (antagonize) the binding of DCL polypeptides to their natural ligand(s).

The invention additionally provides a method of designing an inhibitor of the DCL polypeptides, the method comprising the steps of determining the three-dimensional structure of any such polypeptide, analyzing the three-dimensional structure for the likely binding sites of substrates, synthesizing a molecule that incorporates a predicted reactive site, and determining the polypeptide-inhibiting activity of the molecule.

In a further aspect of the invention, a method is provided for identifying compounds that alter DCL polypeptide activity comprising

(a) mixing a test compound with a polypeptide of the invention; and

(b) determining whether the test compound alters the DCL polypeptide activity of said polypeptide.

In another aspect of the invention, a method is provided identifying compounds that inhibit the binding activity of DCL polypeptides comprising

(a) mixing a test compound with a polypeptide of the invention and a binding partner of said polypeptide; and

(b) determining whether the test compound inhibits the binding activity of said polypeptide.

In alternative embodiments, the binding partner is a natural ligand, which may be an antigen, which in turn may be an/a oligosaccharide, polysaccharide, carbohydrate, glycoprotein, phospholipid, glycolipid, glycosphingolipid and the like; the natural ligand may be selected from the group consisting of bacterial, viral, fungal or protazoan polypeptides, as well as cell membrane-associated polypeptides. A binding partner may alternatively comprise an antibody, either agonistic or antagonistic to DCL activity. Also, a binding partner may comprise a fragment, derivative, fusion protein or peptidomimetic of a DCL natural ligand.

The invention also provides a method for increasing DCL polypeptide activities comprising providing at least one compound selected from the group consisting of the polypeptides of the invention and agonists of said polypeptides. An additional embodiment of the method further comprising increasing said activities in a patient by administering at least one polypeptide of the invention. Agonists may comprise antibodies, isolated DCL polypeptide(s) or fragment(s) thereof, DCL peptide(s) and/or peptidomimetic(s).

DCL polypeptide activities include, but are not limited to, antigen binding, antigen internalization, antigen processing and antigen presentation; antigen presenting cell (APC) activation, APC differentiation, APC maturation, APC homing and APC transmigration; cell to cell interactions including binding and modulation of intracellular signaling pathways in either an excitatory or inhibitory manner; extracellular communication through secretion of soluble factors that act in an autocrine, paracrine and/or endocrine fashion; C-type lectin activity; carbohydrate recognition domain activity; aspartyl protease activity and immunoreceptor tyrosine-based inhibitory-like motif (ITIM) activity. Examples of cells that may bind to APCs expressing DCL polypeptides include cells of the immune system, including T-cells, B-cells, NK cells, as well as precursors thereof.

Further provided by the invention is a method for decreasing one or more of the activities described immediately above, comprising providing at least one antagonist of the polypeptides of the invention; with a preferred embodiment of the method further comprising decreasing said activities in a patient by administering at least one antagonist of the polypeptides of the invention, and with a further preferred embodiment wherein the antagonist is an antibody, an isolated DCL polypeptide or fragment thereof, DCL peptide and/or peptidpmimetic that inhibits the activity of any of said polypeptides.

In other aspects, the invention provides assays utilizing DCL compositions to screen for potential agonists and/or antagonists of DCL activity and/or DCL-associated cellular events. In addition, methods of using DCL polypeptides, polynucleotides, fragments, variants, muteins, fusion proteins, antibodies, binding proteins and the like in the rational design of antagonists and/or agonists thereof are also an aspect of the invention.

The invention additionally provides a method for treating autoimmune disorders, inflammation, cancer, transplantation-associated conditions, and infectious diseases comprising administering at least one compound selected from the group consisting of the polypeptides of the invention and agonists and antagonists of said polypeptides.

### BRIEF DESCRIPTION OF THE FIGURES

FIG. 1 shows the polynucleotide and polypeptide sequences for DCL 1. The ∥ symbol in the polynucleotide sequence denotes exon/intron junction (introns not shown) and underlined regions show the positions of oligonucleotide primers used in PCR reactions. In the polypeptide sequence, italic type indicates predicted transmembrane domains; boxed sequences indicate predicted aspartyl (or acid) protease domains; bold-italic type denotes an ITIM motif; underlined regions indicate C-type lectin domains and bold type indicates putative N-linked glycosylation sites.

FIG. 2 shows the polynucleotide and polypeptide sequences for DCL 2. The same annotations described for FIG. 1 are also employed in FIG. 2.

FIG. 3 shows the polynucleotide and polypeptide sequences for DCL 3. The annotations described for FIG. 1 are employed in FIG. 3.

FIG. 4 shows the polynucleotide and polypeptide sequences for DCL 4. The annotations described for FIG. 1 are employed in FIG. 4.

FIG. 5 shows the polynucleotide and polypeptide sequences for DCL 5. The annotations described for FIG. 1 are employed in FIG. 5.

### DETAILED DESCRIPTION OF THE INVENTION

The present invention is directed to identifying, isolating and characterizing novel members of the calcium-dependent (C-type) lectin family associated with mammalian cells, and in particular, antigen presenting cells of the DC lineage. The present invention provides novel polypeptides having C-type lectin domains that are expressed on murine dendritic cells, herein designated as DCL 1, DCL 2, DCL 3 and DCL 4, as well as splice variants svDCL 2, svDCL 3 and svDCL 4, and a human homologue herein designated DCL 5. For convenience, DCL 1, DCL 2, DCL 3, DCL 4 and DCL 5 (as well as splice variants and homologs) are often referred to collectively as DCL polypeptides. When using the term DCL, it is understood to mean one or more of DCL 1, 2, 3, 4 and/or 5, as well as splice variants svDCL 2, svDCL 3 and svDCL 4, alone or in any combination.

The present invention provides polynucleotides encoding DCL polypeptides and recombinant expression vectors that include polynucleotides encoding DCL polypeptides. The present invention additionally provides methods for isolating DCL polypeptides and methods for producing recombinant DCL polypeptides by cultivating host cells transfected or transformed with the recombinant expression vectors under conditions appropriate for expressing polypeptides of the present invention and recovering the expressed polypeptides.

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which will be limited only by the appended claims.

The term “vector” is used to refer to any molecule (e.g. nucleic acid, plasmid, or virus) used to transfer coding information to a host cell. The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing, if introns are present.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “host cell” is used to refer to a cell that has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “DCL polypeptide fragment” refers to a polypeptide that comprises a truncation at the amino-terminus (with or without a leader sequence) and/or a truncation at the carboxyl-terminus of the polypeptide as set forth in either SEQ ID NOs: 2, 6, 10, 12, 16, 18, 22 and 24. The term “DCL polypeptide fragment” also refers to amino-terminal and/or carboxyl-terminal truncations of DCL polypeptide orthologs, DCL polypeptide derivatives, or DCL polypeptide variants, or to amino-terminal and/or carboxyl-terminal truncations of the polypeptides encoded by DCL polypeptide allelic variants or DCL polypeptide splice variants. DCL polypeptide fragments may result from alternative RNA splicing or from in vivo protease activity. Membrane-bound forms of an DCL polypeptide are also contemplated by the present invention. In preferred embodiments, truncations and/or deletions comprise about 10 amino acids, or about 20 amino acids, or about 50 amino acids, or about 75 amino acids, or about 100 amino acids, or more than about 100 amino acids. The polypeptide fragments so produced will comprise about 25 contiguous amino acids, or about 50 amino acids, or about 75 amino acids, or about 100 amino acids, or about 150 amino acids, or about 200 amino acids. Such DCL polypeptide fragments may optionally comprise an amino-terminal methionine residue. It will be appreciated that such fragments can be used, for example, to generate antibodies to DCL polypeptides.

The term “DCL polypeptide ortholog” refers to a polypeptide from another species that corresponds to DCL polypeptide amino acid sequence as set forth in either SEQ ID NO: 2 or SEQ ID NO: 5. For example, mouse and human DCL polypeptides are considered orthologs of each other.

The term “DCL polypeptide variants” refers to DCL polypeptides comprising amino acid sequences having at least one amino acid sequence substitutions, deletions (such as internal deletions and/or DCL polypeptide fragments), and/or additions (such as internal additions and/or DCL fusion polypeptides) as compared to the DCL polypeptide amino acid sequence set forth in either SEQ ID NO: 2 or SEQ ID NO: 5 (with or without a leader sequence). Variants may be naturally occurring (e.g., DCL polypeptide allelic variants, DCL polypeptide orthologs, and DCL polypeptide splice variants) or artificially constructed. Such DCL polypeptide variants may be prepared from the corresponding nucleic acid molecules having a DNA sequence that varies accordingly from the DNA sequence as set forth in either SEQ ID NO: 1 or SEQ ID NO: 4. In preferred embodiments, the variants have from 1 to 3, or from 1 to 5, or from 1 to 10, or from 1 to 15, or from 1 to 20, or from 1 to 25, or from 1 to 50, or from 1 to 75, or from 1 to 100, or more than 100 amino acid substitutions, insertions, additions and/or deletions, wherein the substitutions may be conservative, or non-conservative, or any combination thereof.

The term “DCL polypeptide derivatives” refers to the polypeptide as set forth in either DCL 1 (SEQ ID NO:2), DCL 2 (SEQ ID NO:6), DCL 3 (SEQ ID NO:12) and DCL 4 (SEQ ID NO:22), as well as splice variants svDCL 2 (SEQ ID NO:10), svDCL 3 (SEQ ID NO:16) and svDCL 4 (SEQ ID NO:22), and a human homologue, herein designated DCL 5 (SEQ ID NO:24); DCL polypeptide fragments; DCL polypeptide orthologs; or DCL polypeptide variants; as defined herein, that have been chemically modified. The term “DCL polypeptide derivatives” also refers to the polypeptides encoded by DCL polypeptide allelic variants or DCL polypeptide splice variants, as defined herein, that have been chemically modified.

The term “mature DCL polypeptide” refers to a DCL polypeptide lacking a leader sequence. A mature DCL polypeptide may also include other modifications such as proteolytic processing of the amino-terminus (with or without a leader sequence) and/or the carboxyl-terminus, cleavage of a smaller polypeptide from a larger precursor, N-linked and/or O-linked glycosylation, and the like. Exemplary mature DCL polypeptides are depicted by the amino acid sequences of DCL 1 (SEQ ID NO:2), DCL 2 (SEQ ID NO:6), DCL 3 (SEQ ID NO:12) and DCL 4 (SEQ ID NO:22), as well as splice variants svDCL 2 (SEQ ID NO:10), svDCL 3 (SEQ ID NO:16) and svDCL 4 (SEQ ID NO:22), and a human homologue, herein designated DCL 5 (SEQ ID NO:24).

The term “DCL fusion polypeptide” refers to a fusion of one or more amino acids (such as a heterologous protein or peptide) at the amino- or carboxyl-terminus of the polypeptide as set forth in DCL 1 (SEQ ID NO:2), DCL 2 (SEQ ID NO:6), DCL 3 (SEQ ID NO:12) and DCL 4 (SEQ ID NO:22), as well as splice variants svDCL 2 (SEQ ID NO:10), svDCL 3 (SEQ ID NO:16) and svDCL 4 (SEQ ID NO:22), and a human homologue, herein designated DCL 5 (SEQ ID NO:24), DCL polypeptide fragments, DCL polypeptide orthologs, DCL polypeptide variants, or DCL derivatives, as defined herein. The term “DCL fusion polypeptide” also refers to a fusion of one or more ammo acids at the amino- or carboxyl-terminus of the polypeptide encoded by DCL polypeptide allelic variants or DCL polypeptide splice variants, as defined herein.

The term “biologically active DCL polypeptides” refers to DCL polypeptides having at least one DCL activity characteristic of the polypeptide comprising the amino acid sequence of DCL 1 (SEQ ID NO:2), DCL 2 (SEQ ID NO:6), DCL 3 (SEQ ID NO:12), DCL 4 (SEQ ID NO:22) and DCL 5 (SEQ ID NO:24), as well as splice variants svDCL 2 (SEQ ID NO:10), svDCL 3 (SEQ ID NO:16) and svDCL 4 (SEQ ID NO:22). Examples of DCL activities include, but are not limited to, antigen binding, antigen internalization, antigen processing and antigen presentation; antigen presenting cell (APC) activation, APC differentiation, APC maturation, APC homing and APC transmigration; cell to cell interactions including binding and modulation of intracellular signaling pathways in either an excitatory or inhibitory manner; extracellular communication through secretion of soluble factors that act in an autocrine, paracrine and/or endocrine fashion; C-type lectin activity; carbohydrate recognition domain activity; aspartyl protease activity and immunoreceptor tyrosine-based inhibitory-like motif (ITIM) activity. Examples of cells that may bind to APCs expressing DCL polypeptides include cells of the immune system, including T-cells, B-cells, NK cells, as well as precursors thereof.

In addition, a DCL polypeptide may be active as an immunogen; that is, the DCL polypeptide contains at least one epitope to which antibodies may be raised.

The term “naturally occurring” or “native” when used in connection with biological materials such as nucleic acid molecules, polypeptides, host cells, and the like, refers to materials which are found in nature and are not manipulated by man. Similarly, “non-naturally occurring” or “non-native” as used herein refers to a material that is not found in nature or that has been structurally modified or synthesized by man.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, may be maintained transiently as an episomal element without being replicated, or may replicate independently as a plasmid. A cell is considered to have been stably transformed when the DNA is replicated with the division of the cell.

A “peptibody” refers to molecules comprising an Fc domain and at least one peptide. Such peptibodies may be multimers or dimers or fragments thereof, and they may be derivatized. Peptibodies are described in greater detail in WO 00/24782 and WO 01/83525, which are incorporated herein by reference in their entirety. The peptide may be from the amino acid sequence of DCL 1 (SEQ ID NO:2), DCL 2 (SEQ ID NO:6), DCL 3 (SEQ ID NO:12) and DCL 4 (SEQ ID NO:22), as well as splice variants svDCL 2 (SEQ ID NO:10), svDCL 3 (SEQ ID NO:16) and svDCL 4 (SEQ ID NO:22), and a human homologue, herein designated DCL 5 (SEQ ID NO:24).

A “peptide,” as used herein refers to molecules of 1 to 40 amino acids. Alternative embodiments comprise molecules of 5 to 20 amino acids. Exemplary peptides may comprise portions of the extracellular domain of naturally occurring molecules or comprise randomized sequences of DCL 1 (SEQ ID NO:2), DCL 2 (SEQ ID NO:6), DCL 3 (SEQ ID NO:12) and DCL 4 (SEQ ID NO:22), as well as splice variants svDCL 2 (SEQ ID NO:10), svDCL 3 (SEQ ID NO:16) and svDCL 4 (SEQ ID NO:22), and a human homologue, herein designated DCL 5 (SEQ ID NO:24).

The term “randomized” as used to refer to peptide sequences refers to fully random sequences (e.g., selected by phage display methods or RNA-peptide screening) and sequences in which one or more residues of a naturally occurring molecule is replaced by an amino acid residue not appearing in that position in the naturally occurring molecule. Exemplary methods for identifying peptide sequences include phage display, E. coli display, ribosome display, RNA-peptide screening, chemical screening, and the like.

The term “Fc domain” encompasses native Fc and Fc variant molecules and sequences as defined below. As with Fc variants and native Fc's, the term “Fc domain” includes molecules in monomeric or multimeric form, whether digested from whole antibody or produced by other means.

The term “native Fc” refers to molecule or sequence comprising the sequence of a non-antigen-binding fragment resulting from digestion of whole antibody, whether in monomeric or multimeric form. The original immunoglobulin source of the native Fc is preferably of human origin and may be any of the immunoglobulins, although IgG1 and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “Fc variant” refers to a molecule or sequence that is modified from a native Fc but still comprises a binding site for the salvage receptor, FcRn. International applications WO 97/34631 (published 25 Sep. 1997) and WO 96/32478 describe exemplary Fc variants, as well as interaction with the salvage receptor, and are hereby incorporated by reference in their entirety. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Pc variants are described in further detail hereinafter.

A “peptidomimetic” is a peptide analog that displays more favorable pharmacological properties than their prototype native peptides, such as a) metabolic stability, b) good bioavailability, c) high receptor affinity and receptor selectivity, and d) minimal side effects. Designing peptidomimetics and methods of producing the same are known in the art (see for example, U.S. Pat. Nos. 6,407,059 and 6,420,118). Peptidomimetics may be derived from the binding site of the extracellular domain of DCL 1-5 and splice variants svDCL 2, svDCL 3 and svDCL 4. In alternative embodiments, a peptidomimetic comprises non-peptide compounds having the same three-dimensional structure as peptides derived from DCL 1-5 and splice variants svDCL 2, svDCL 3 and svDCL 4, or compounds in which part of a peptide from the molecules listed above is replaced by a non-peptide moiety having the same three-dimensional structure.

A “mimotope” is defined herein as peptide sequences that mimic binding sites on proteins (see, Partidos, CD, et al., Combinatorial Chem & High Throughput Screening, 2002 5:15-27). A mimotope may have the capacity to mimic a conformationally-dependent binding site of a protein. The sequences of these mimotopes do not identify a continuous linear native sequence or necessarily occur in a naturally-occurring protein. Mimotpes and methods of production are taught in U.S. Pat. No. 5,877,155 and U.S. Pat. No. 5,998,577, which are incorporated by reference in their entireties.

The term “acidic residue” refers to amino acid residues in D- or L-form having sidechains comprising acidic groups. Exemplary acidic residues include D and E.

The term “amide residue” refers to amino acids in D- or L-form having sidechains comprising amide derivatives of acidic groups. Exemplary residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.,

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L and norleucine (Nle).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

The term “subject” as used herein, refers to mammals. For example, mammals contemplated by the present invention include humans; primates; pets of all sorts, such as dogs, cats, etc.; domesticated animals, such as, sheep, cattle, goats, pigs, horses and the like; common laboratory animals, such as mice, rats, rabbits, guinea pigs, etc.; as well as captive animals, such as in a zoo or free wild animals. Throughout the specification, the term host is used interchangeably with subject.

As used herein the singular forms “a”, “and”, and “the” include plural referents unless the context clearly dictates otherwise. Thus, for example, reference to “an immunization” includes a plurality of such immunizations and reference to “the cell” includes reference to one or more cells and equivalents thereof known to those skilled in the art, and so forth. All technical and scientific terms used herein have the same meaning as commonly understood to one of ordinary skill in the art to which this invention belongs unless clearly indicated otherwise.

As used herein, a dendritic cell, or DC, refers to any member of a diverse population of phenotypically and/or morphologically similar cell types found in lymphoid or non-lymphoid tissues. DCs are a class of “professional” antigen presenting cells, and have a high capacity for sensitizing MHC-restricted T cells. Depending upon their lineage and stage of maturation, DCs may be recognized by function, or by phenotype, particularly by cell surface phenotype. These cells are characterized by their distinctive morphology, phagocytic/endocytotic capacity, high levels of surface MHC-class II expression and ability to present antigen to T cells, particularly to naive T cells (Banchereau, et al., Annu. Rev. Immunol., 18:767-811, 2000 and U.S. Pat. No. 6,274,378, incorporated herein by reference for its description of such cells). For illustrative purposes only, DCs described herein may be characterized by veil-like projections and expression of the cell surface markers CD1a⁺, CD4⁺, CD86⁺, or HLA-DR⁺. Mature DCs are typically CD11c⁺, while precursors of DCs include those having the phenotype CD11c⁻, IL-3Rα^{low}; and those that are CD11c⁻L-3Rα^{high}. Treatment with GM-CSF in vivo preferentially expands CD11b^{high}, CD11c^{high} DC, while Flt-3 ligand has been shown to expand CD11c⁺ IL-3Rα^{low} DC, and CD11c⁻IL-3Rα^{high} DC precursors. The DCs expressing C-type lectins of the present invention may be immature or mature dendritic cells of the lymphoid and/or myeloid lineage. Functionally, dendritic cells maybe identified by any convenient assay for determination of antigen presentation. Such assays may include testing the ability to stimulate antigen-primed or naive T cells by presentation of a test antigen, following by determination of T cell proliferation, release of IL-2, and the like.

A C-type lectin, as used herein, refers to any of the Ca⁺⁺-dependent binding proteins having affinity for and the capacity to bind to carbohydrate moieties, as well as other attributes well known in the art, which is referred to herein as “C-type lectin activity.” C-type lectins also include collectins, selectins and the C-type lectin superfamily of the immune system, as reviewed in Weis, W. I., Immunol. Rev., 163:19-34, 1998 and Feizi, T., Immunol. Rev., 173:79-88, 2000).

Identifying genes that are upregulated in DCs in response to external stimuli, such as bacterial antigens and pro-inflammatory cytokines, may shed light on how the immune system responds to varying types of stimuli. Generally speaking, DCs mature in response to bacterial lipopolysaccharide (LPS) and CpG DNA, TNF-α or CD40-Ligand, which represent pathogens, endogenous inflammatory signals or T cell feedback signals, respectively. Following in vitro or in vivo exposure to bacterial antigens, DCs undergo maturation by one of two signaling pathways: via the ERK kinase pathway, which allows for DC survival, or via the NF-κB signaling pathway, which is characterized by increased expression of costimulatory and MHC-class II molecules, release of chemokines and migration culminating in high T cell stimulatory capacity and IL-12 release. Bacterial LPS is one of the major molecules recognized by the innate immune system (Verhasselt, H., et al., J. Immunol. 158:2919-25, 1997). Ligation of membrane-associated CD14 by LPS complexes and soluble LPS-binding protein lead to pro-inflammatory signals, such as TNF and IL-1 secretion, which increase the turnover of local APCs as well as recruitment of precursor cells at the site of tissue damage. Also, Toll-like receptor-2 (TLR2) has been shown to be a signaling receptor activated by LPS in a response that is dependent on LPS-binding protein and is enhanced by CD14 (Yang, R. B., et al., Nature 395:284-88, 1998). Toll-like receptor-4 (TLR4) transduces intracellular signaling in LPS responses leading to NF-κB activation and TLR4-deficient mice are hyporesponsive to LPS (Brightbill, H. D., et al., Science 285:732-36, 1999). In addition, TLR2, but not TLR4, mediates responses elicited by components of gram-positive bacteria, such as peptidoglycan and lipoteichoic acid (Yoshimura, A., et al., J. Immunol. 163:1-5, 1999; Schwander, R., et al., J. Biol. Chem. 274:17406-9, 1999). Other Toll-like receptors include, Toll-like receptor-5 (TLR5), which recognizes and is activated by bacterial flagellin (Hayashi, F., et al., Nature 408:740-745, 2000), and Toll-like receptor-9 (TLR9), which recognizes and is activated by hypomethylated CpG DNA motifs (Hemmi H., et al., Nature 410:1099-1103, 2001)

Different antigenic stimuli have profound effects on DC biology that may influence the immune system as a whole. Generally speaking, IL-12-producing myeloid DCs prime Th1 responses, whereas lymphoid DCs that produce Interferon α and/or β, prime Th2 responses, which are driven by IL-4 produced by activated T cells (Kalinsky, P., et al., Immunol Today 20:561-67, 1999). Myeloid DCs produce IL-12 in response to pathogens such as bacteria, viruses and mycoplasmas, but fail to do so in response to other maturation stimuli such as TNF-α, IL-1, cholera toxin or FasL. IL-12 production can be potently induced by CD40L, which is expressed at high levels on activated memory T cells (Cella, M., J. Exp. Med. 184:747-52, 1996). However, systemic stimulation with LPS leads to a paralysis of IL-12 production (Reis e Sousa, C., et al., Immunity 11:637-47, 1999). Furthermore, various cytokines present in peripheral tissues during the induction of DC maturation can also modulate IL-12 production. For example, IFN-γ and IL-4 enhance IL-12 production induced by LPS or CD40L, whereas IL-10 has a suppressive effect, and TGF-β has also been shown to inhibit the response to LPS while augmenting the response to CD40L.

Determining the effect of antigenic stimuli, such as LPS, on DC biology may provide insights into antigen binding and uptake, antigen processing and presentation, the activation, differentiation, maturation, homing and transmigration of antigen presenting cells, as well as cell to cell interactions with various cells of the immune system, such as T- and B-cells. Towards this end, murine DC populations were treated with various agents, such as LPS and IFN-α, to determine differential expression of DC-associated genes in response to those agents. Through this type of analysis, four novel murine genes that encode polypeptides having, inter alia, C-type lectin domains were discovered, which are referred to as Dendritic Cell C-type Lectins 1 through 4 (DCL 1-4), as well as splice variants thereof. Additionally, a novel human homologue of the DCL 14 polypeptides has been discovered and is referred to as DCL 5 (DCL 1 (SEQ ID NO:2), DCL 2 (SEQ ID NO:6), DCL 3 (SEQ ID NO:12) and DCL 4 (SEQ ID NO:22), as well as splice variants svDCL 2 (SEQ ID NO:10), svDCL 3 (SEQ ID NO:16) and svDCL 4 (SEQ ID NO:22), and a human homologue, herein designated DCL 5 (SEQ ID NO:24)).

Bone marrow (BM) cells were isolated from C57BL/10 mice and cultured under conditions essentially as described in Example 1. BM cells were cultured in Flt3-ligand for nine days. The cultures were stimulated for 4 hours with the following stimuli/conditions: (a) 10 ng/ml recombinant murine GM-CSF, 1000 U/ml human; (b) 500 U/ml IFN-alpha/D (Genzyme, Cambridge, Mass.); (c) 1 μg/ml Escherichia coli (E coli)(0217:B8)-derived LPS (Difco, Detroit, Mich.) and (d) no stimulus. After 4 hr expossure to the stimuli, the cells were lysed and the RNA isolated using methods well known in the art. In a separate set of experiments, mice were treated with Flt3-ligand or pegylated GM-CSF prior to harvesting in order to increase the number of DCs.

The preparation of the target RNAs and hybridization to the microarray chips was performed essentially as described in the Affymetrix protocols (Affymetrix Corp., Santa Clara, Calif.), which are incorporated herein by reference. Briefly, the target sample was prepared using 10 ug of total RNA, which was first converted to single-stranded cDNA using Superscript II™ reverse transcriptase (Gibco BRL Life Technologies) and a primer encoding the bacteriophage T7 RNA polymerase promoter. The single-stranded cDNA was then converted to double-stranded cDNA. The T7 promoter was used to generate a labeled cRNA target in a reaction containing T7 RNA polymerase and biotinylated nucleotide triphosphates. After purification, the cRNA was chemically fragmented to an average length of 50-200 bases and hybridized overnight at 45° C. to Affymetrix Gene Chips™. After hybridization, the chips were processed in the Affymetrix fluidics station where they were washed, stained with streptavidin phycoerythrin (SAPE), probed with biotinylated goat anti-streptavidin, and finally, a second round of SAPE.

Polynucleotides Encoding DCL Polypeptides

The present invention provides novel polypeptides of the calcium-dependent lectin family that are expressed on murine dendritic cells, herein designated as DCL 1, DCL 2, DCL 3 and DCL 4, as well as splice variants svDCL 2, svDCL 3 and svDCL 4, and a human homologue herein designated DCL 5. Such proteins are substantially free of contaminating endogenous materials and, optionally, without associated native-pattern glycosylation. Derivatives of DCL polypeptides within the scope of the invention also include various structural forms of the primary protein which retain biological activity. Due to the presence of ionizable amino and carboxyl groups, for example, DCL protein may be in the form of acidic or basic salts, or may be in neutral form. Individual amino acid residues may also be modified by oxidation or reduction.

Gene microarray technology provides a tool to study differential gene expression across different mouse dendritic cell subpopulations derived under a number of different stimulation conditions. As described above, the hybridization signals from DC stimulated with LPS were compared with the signals from DC stimulated with IFN-α, as well as those from DC stimulated with GM-CSF. Gene expression upregulated by LPS, but unaltered by IFN-α or GM-CSF, were identified. Analysis of the gene sequences that correspond to the identified signals was performed. One such gene identified was represented in the Affymetrix data as GenBank accession no. AA389977 and NCBI Unigene entry MM.3443. From the NCBI Unigene site, nine GenBank accessions, including AA389977, were listed as corresponding to this same Unigene entry. The sequences from these nine entries were assembled, and the resulting ‘contig’ was compared using the BLAST algorithm to public database protein sequences. From this comparison, the contig was revealed to encode a putative protein with homology to molecules characterized as “C-type lectins.”

Since the assembled EST contig comparison with these known C-type lectins predicted that the assembly encoded an incomplete (missing the 5′ end) sequence, this same EST assembly was used in a BLAST comparsion with mouse genomic sequences contained within the Celera™ proprietary database to obtain the genomic counterpart. Searching candidate mouse genomic sequences for the specific coding regions corresponding to a C-type lectin open reading frame revealed that multiple genes were contained within a single large mouse genomic fragment (over 400,000 bp). Further analysis showed that there are nine or more closely related genes in the mouse, including the following four novel sequences: DCL 1 (SEQ ID NO:1 and the corresponding amino acid sequence provided in SEQ ID NO:2), DCL 2 (SEQ ID NO:5 and the corresponding amino acid sequence provided in SEQ ID NO:6), DCL 3 (SEQ ID NO:11 and the corresponding amino acid sequence provided in SEQ ID NO:12) and DCL 4 (SEQ ID NO:17 and the corresponding amino acid sequence provided in SEQ ID NO:18). Given their close chromosomal proximity to each other and their high degree of homology, it is likely that these genes arose through gene duplication events.

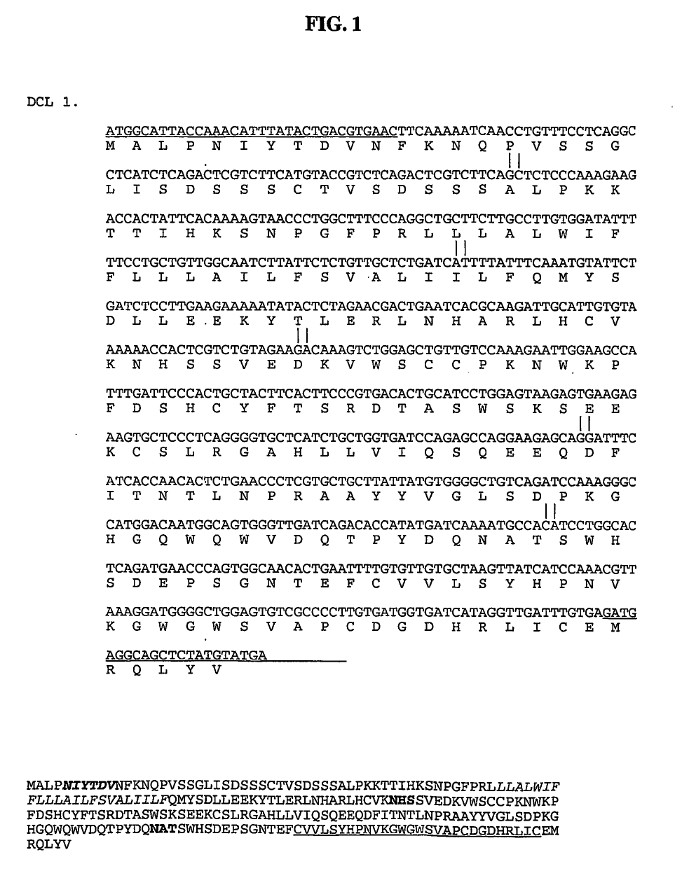

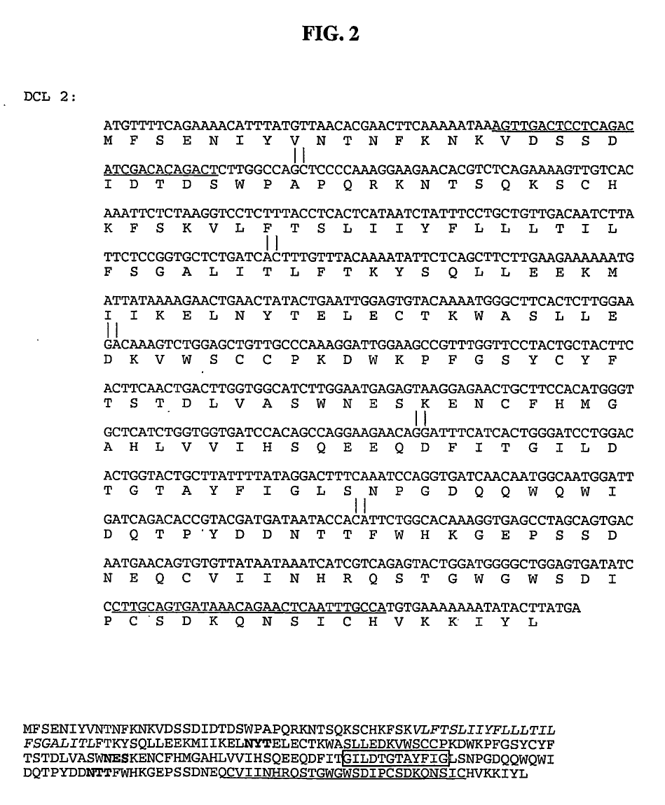

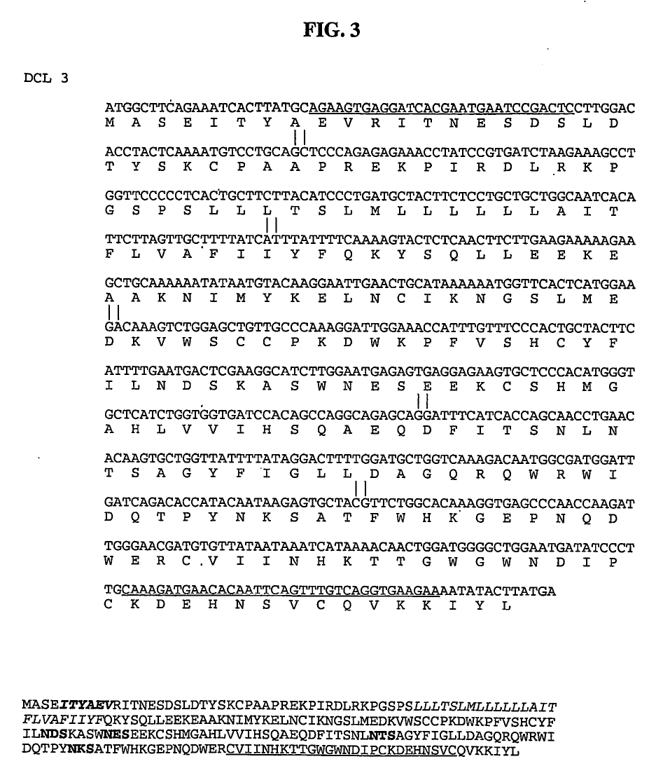

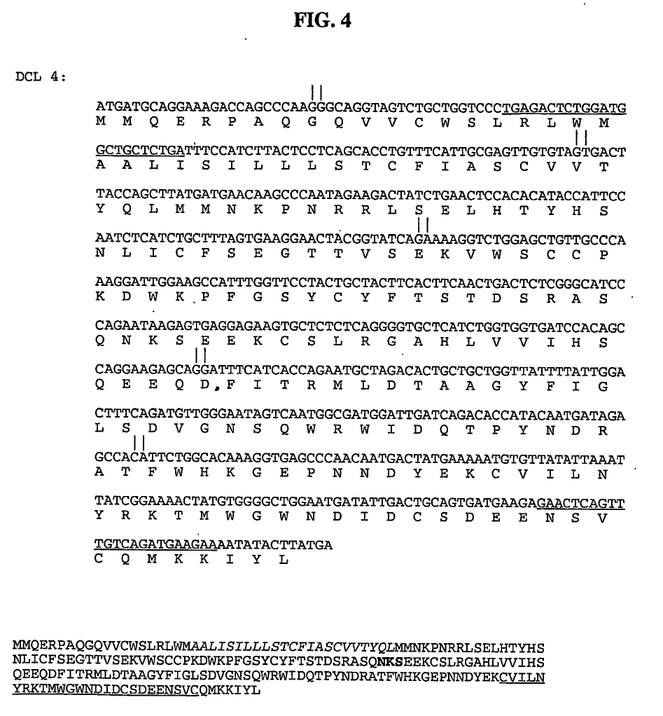

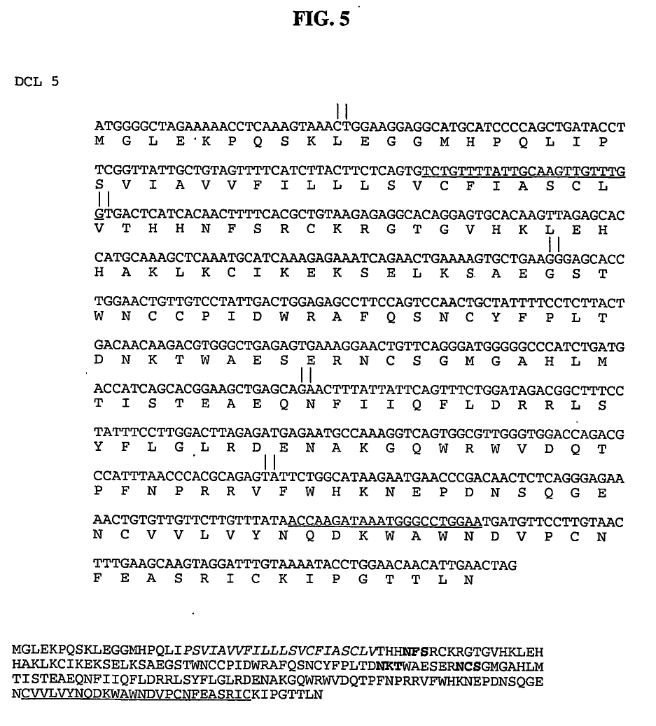

To predict the existence of multiple family members, mouse genomic contigs, which were determined to encode the sequence corresponding to DCL1 and related sequences, were examined by comparing the amino acid sequences of two known family members, dectin 2 (Ariizumi, K., et al., supra) and DCIR, which is also referred to as dcmp1 (Bates, E., et al., supra) with all 6 possible translated reading frames of the genomic contigs, using the GCG program TFASTA. Iterative TFASTA analyses and manual examination of the outputs led to the realization that a large number of related genes existed. At this time, it is thought that there are nine different closely related mouse genes, with five corresponding human genes. Using the TFASTA program, sequence maps of the mouse genomic regions and an understanding of the canonical sequences of exon/intron junctions in mammalian DNA, the open reading frames and intron/exon boundaries were predicted for the DCL 1-5 polypeptides. Using this sequence information, unique oligonucleotide pairs specific to each gene's 5′ and 3′ coding region were designed and synthesized. Specifically, SEQ ID NOs:3 and 4 are the sense and antisense oriented PCR primers, respectively, for DCL 1 (see FIG. 1); SEQ ID NOs:7 and 8 are the sense and antisense oriented PCR primers, respectively, for DCL 2 (see FIG. 2); SEQ ID NOs:13 and 14 are the sense and antisense oriented PCR primers, respectively, for DCL 3 (see FIG. 3); SEQ ID NOs:19 and 20 are the sense and antisense oriented PCR primers, respectively, for DCL 4 (see FIG. 4); and SEQ ID NOs:25 and 26 are the sense and antisense oriented PCR primers, respectively, for DCL (see FIG. 5). These primer pairs were added to PCR mixes containing templates from a large collection of human and mouse tissue-specific cDNAs (Clontech, Palto Alto, Calif.), and PCRs were performed. Amplimers of the predicted sizes were obtained from these reactions. These fragments were gel purified and submitted for DNA sequence analysis, which demonstrated that the determined cDNA sequences were identical to the predicted sequences for all five novel DCL molecules. In addition, smaller amplimers were sequenced and found to encode alternate splice forms, namely svDCL 2, svDCL 3 and svDCL 4.

Throughout the following discussion, the amino acid designations for defined motifs and/or polypeptide regions and/or signature sequences are inclusive. Those skilled in the art will recognize that naturally occurring variants, such as allelic variant, may vary in the numbering of these regions and therefore may differ from that predicted by computer analysis. Thus, the amino acid designation for the beginning and ending of a region or motif may vary from 1 to 5 amino acids from the ascribed numbering. C-type lectin domains and internal signature patterns were determined using the GCG program MOTIFS (PROSITE Dictionary of Protein Sites and Patterns). N-linked glycosylation sites are defined herein as Asn-X-Ser/Thr, where X is any amino acid except proline. Predictions were made using the Transmembrane Hidden Markov Model (TMHMM) prediction tool at http://www.cbs.dtu.dk/services/, and the C-type lectin and aspartyl protease domains were predicted using the GCG program MOTIFS, which uses the PROSITE Dictionary of protein patterns. Other programs used by those skilled in the art of sequence comparison can also be used, such as, for example, the BLASTN program version 2.0.9, available for use via the National Library of Medicine website www.ncbi.nlm.nih.gov/gorf/wblast2.cgi, or the UW-BLAST 2.0 algorithm. Standard default parameter settings for UW-BLAST 2.0 are described at the following Internet site: sapiens.wustl.edu/blast/blast/#Features. Immunoreceptor tyrosine-based inhibitory motifs (ITIMs) were predicted using the established consensus sequence.

The cDNA sequence for DCL 1 is provided in SEQ ID NO:1 and comprises a 738 bp polynucleotide having an initiation codon, 5 exon/intron splice junction sites and a stop codon at nucleotides 736-738, as depicted in FIG. 1. DCL 1 has been mapped to murine chromosome 6. The full-length DCL 1 polypeptide sequence (SEQ ID NO:2) comprises a 245 amino acid open reading frame (ORF) having an amino-terminus intracellular region essentially spanning amino acids 1-53, a transmembrane region essentially spanning amino acids 54-76 and an extracellular region essentially spanning amino acids 77-245. The extracellular region has a number of putative N-linked glycosylation sites found approximately at amino acids 102-104 and 195-197. DCL 1 has a characteristic C-type lectin domain having a representative signature sequence spanning approximately amino acids 211-238 and an immunoreceptor tyrosine-based inhibitory-like motif (ITIM) at approximately amino acids 5-10. Soluble DCL 1 comprises the extracellular domain (residues 77-245 of SEQ ID NO:2) or a fragment thereof.

The cDNA sequence for DCL 2 is provided in SEQ ID NO:5 and comprises a 714 bp polynucleotide having an initiation codon, 5 exon/intron splice junction sites and a stop codon at nucleotides 712-714, as depicted in FIG. 2. DCL 2 has been mapped to murine chromosome 6. The full-length DCL 2 polypeptide sequence (SEQ ID NO:6) comprises a 237 amino acid ORF having an amino-terminus intracellular region essentially spanning amino acids 144, a transmembrane region essentially spanning amino acids 45-68 and an extracellular region essentially spanning amino acids 69-237. The extracellular region has a number of putative N-linked glycosylation sites found approximately at amino acids 86-88, 130-132 and 188-190. DCL 2 also has a predicted aspartyl (or acid) protease domain spanning approximately amino acids 157-168, as well as a characteristic C-type lectin domain having a representative signature sequence spanning approximately amino acids 204-230. Soluble DCL 2 comprises the extracellular domain (residues 69-237 of SEQ ID NO:6) or a fragment thereof.

In addition, DCL 2 has a truncated splice variant isoform referred to as svDCL 2 wherein exon 3 has been deleted (cDNA sequence provided in SEQ ID NO:9 and corresponding amino acid sequence provided in SEQ ID NO:10). The svDCL 2 cDNA sequence comprises a 612 bp fragment having an initiation codon, 4 exon/intron splice junction sites and a stop codon at nucleotides 610-612. The full-length svDCL 2 polypeptide sequence comprises a 203 amino acid ORF having an amino-terminus intracellular region essentially spanning amino acids 144, a transmembrane region essentially spanning amino acids 45-67 and an extracellular region essentially spanning amino acids 68-203. The extracellular region has a number of putative N-linked glycosylation sites at amino acids 96-98 and 154-156. svDCL 2 also has a predicted aspartyl (or acid) protease domain spanning approximately amino acids 123-134, as well as a characteristic C-type lectin domain having a representative signature sequence spanning approximately amino acids 170-196. Soluble svDCL 2 comprises the extracellular domain (residues 68-203 of SEQ ID NO:10) or a fragment thereof.

The cDNA sequence for DCL 3 is provided in SEQ ID NO:11 and comprises a 711 bp polynucleotide having an initiation codon, 5 exon/intron splice junction sites and a stop codon at nucleotides 709-711, as depicted in FIG. 3. DCL 3 has been mapped to murine chromosome 6. The full-length DCL 3 polypeptide sequence (SEQ ID NO:12) comprises a 236 amino acid ORF having an amino-terminus intracellular region essentially spanning amino acids 1-44, a transmembrane region essentially spanning amino acids 45-69 and an extracellular region essentially spanning amino acids 70-236. The extracellular region has a number of putative N-linked glycosylation sites at approximately amino acids 123-125, 130-132, 160-162 and 136-138. DCL 3 has a characteristic C-type lectin domain having a representative signature sequence spanning approximately amino acids 24-229. Soluble DCL 3 comprises the extracellular domain (residues 70-236 of SEQ ID NO:12) or a fragment thereof.

DCL 3 has a truncated splice variant isoform referred to as svDCL 3 wherein exons 4 and 5 are deleted (cDNA sequence provided in SEQ ID NO:15 and corresponding partial amino acid sequence provided in SEQ ID NO:16). The svDCL 3 cDNA sequence comprises a 443 bp fragment having an initiation codon, 3 exon/intron splice junction sites and a number of termination sequences, such as at nucleotides 349-351. One isoform of the predicted svDCL 3 polypeptide sequence comprises a 116 amino acid ORF having an amino-terminus intracellular region essentially spanning amino acids 1-45, a transmembrane region essentially spanning amino acids 46-69 and an extracellular region essentially spanning amino acids 70-116. The extracellular region has an N-linked glycosylation site at amino acids 95-97 and an immunoreceptor tyrosine-based inhibitory motif at approximately amino acids 5-10. Soluble svDCL 3 comprises the extracellular domain (residues 70-116 of SEQ ID NO:16) or a fragment thereof.

The cDNA sequence for DCL 4 is provided in SEQ ID NO:17 and comprises a 627 bp polynucleotide having an initiation codon, 5 exon/intron splice junction sites and a stop codon at nucleotides 625-627, as depicted in FIG. 4. DCL 4 has been mapped to murine chromosome 6. The full-length DCL 4 polypeptide sequence (SEQ ID NO:18) comprises a 208 amino acid ORF having an amino-terminus intracellular region essentially spanning amino acids 1-20, a transmembrane region essentially spanning amino acids 2143 and an extracellular region essentially spanning amino acids 44-208. The extracellular region has a putative N-linked glycosylation site at approximately amino acids 102-104. DCL 4 also has a characteristic C-type lectin domain having a representative signature sequence spanning approximately amino acids 176-201. Soluble DCL 4 comprises the extracellular domain (residues 44-208 of SEQ ID NO:18) or a fragment thereof.

DCL 4 has a truncated splice variant isoform referred to as svDCL 4 wherein exon 4 is deleted (cDNA sequence provided in SEQ ID NO:21 and corresponding partial amino acid sequence provided in SEQ ID NO:22). The svDCL 4 cDNA sequence comprises a 472 bp fragment having an initiation codon, 4 exon/intron splice junction sites and a number of termination sequences, such as at nucleotides 283-285. One isoform of the predicted svDCL 4 polypeptide sequence comprises a 94 amino acid ORF having an amino-terminus intracellular region essentially spanning amino acids 1-19, a transmembrane region essentially spanning amino acids 2042 and an extracellular region essentially spanning amino acids 42-94. Soluble svDCL 4 comprises the extracellular domain (residues 42-94 of SEQ ID NO:16) or a fragment thereof.

A human homologue to the DCL polypeptides was also discovered and is referred to as DCL 5. The cDNA sequence is provided in SEQ ID NO:23 with the determined amino acid sequence provided in SEQ ID NO:24. The DCL 5 polynucleotide sequence comprises a 648 bp polynucleotide having an initiation codon, 5 exon/intron splice junction sites and a stop codon at nucleotides 646-648, as depicted in FIG. 5. DCL 5 has been mapped to human chromosome 12. The full-length DCL 5 polypeptide sequence (SEQ ID NO:24) comprises a 215 amino acid ORF having an amino-terminus intracellular region essentially spanning amino acids 1-19, a transmembrane region essentially spanning amino acids 2041 and an extracellular region essentially spanning amino acids 42-215. The extracellular region has a number of putative N-linked glycosylation sites at approximately amino acids 45-47, 102-104 and 111-113. DCL 5 also has a characteristic C-type lectin domain having a representative signature sequence spanning approximately amino acids 182-207. Soluble DCL 5 comprises the extracellular domain (residues 42-215 of SEQ ID NO:24) or a fragment thereof.

DCL 1-5 are characterized as members of the calcium-dependent lectin family and as type II membrane proteins. DCL 1-5 share homology to other C-type lectin family members such as the Dendritic Cell Immunoreceptor (DCIR), a type II glycoprotein with homology to the macrophage lectin and hepatic asialoglycoprotein receptors, which is believed to play a particular role in directing the ontogeny and/or the Ag-handling potential of DCs for initiation of specific immunity (Bates, E., et al., J. Immunol. 163:1973-83, 1999); DC-associated C-type lectins (Dectin-1 and 2), which are thought to be involved in T-cell binding and delivering T-cell co-stimulatory signals (Ariizumi, K., et al., J. Biol. Chem., 275:20157-167, 2000 and Ariizumi, K., et al., J. Biol. Chem., 275:11957-963, 2000, respectively); and Langerhans cell-specific C-type lectin (Langerin), which is thought to be an endocytotic receptor that induces formation of Birbeck granules (Valladeau, J., Immunity 12:71-81, 2000).

Family members of type II proteins having C-type lectin domains with a single carbohydrate recognition domain at the carboxy terminus include cell surface receptors, such as hepatic asialoglycoprotein receptors 1 and 2 and the macrophage lectin, which binds oligosaccharide groups, and are involved in ligand internalization and uptake of antigen. Therefore, DCL polypeptides are likely to bind oligosaccharide groups and are involved in ligand internalization and uptake of antigen. Furthermore, DCL polypeptides are likely to be involved in cell to cell interaction and communication, such as binding and initiation of intracellular signaling pathways.

The finding that several of the novel polypeptides have the combination of a protease and lectin function is unique. Aspartyl proteases have been associated with activity in intracellular vessicles, as well as associated with cell surface membranes.

DCL1 and DCL 3 also have and at least one immunoreceptor tyrosine-based inhibitory motif (ITIM). Many receptors that mediate positive signaling have cytoplasmic tails containing sites of tyrosine phosphatase phosphorylation known as immunoreceptor tyrosine-based activation motifs (ITAM). A common mechanistic pathway for positive signaling involves the activation of tyrosine kinases, which phosphorylate sites on the cytoplasmic domains of the receptors and on other signaling molecules. Once the receptors are phosphorylated, binding sites for signal transduction molecules are created which initiate the signaling pathways and activate the cell. The inhibitory pathways involve receptors having immunoreceptor tyrosine based inhibitory motifs (ITIM) which, like the ITAMs, are phosphorylated by tyrosine kinases. Receptors having ITIM motifs are involved in inhibitory signaling, which block signaling by removing tyrosine from activated receptors or signal transduction molecules (Renard et al., Immun Rev 155:205-221, 1997). ITIMs have the consensus sequence I/VxYxxL/V (SEQ ID NO:28), and are found in the cytoplasmic portions of diverse signal transduction proteins of the immune system, many of which belong to the Ig superfamily or to the family of type II dimeric C-lectins (see Renard et al., 1997, supra). Proteins that contain ITIMs include the “killer cell Ig-like receptors,” or “KIRs,” and some members of the leukocyte Ig-like receptor or “LIR” family of proteins (Renard et al., 1997, supra; Cosman et al., Immunity 7:273-82, 1997; Borges et al., J. Immunol 159:5192-96, 1997). Signal transduction by an ITIM is believed to downregulate targeted cellular activities, such as expression of cell surface proteins. Renard et al. propose that the regulation of complex cellular functions is fine-tuned by the interplay of ITIM-mediated inhibitory signal transduction and activation of the same functions by a 16-18 amino acid activitory motif, or “ITAM” sequence that is present in other proteins. CD22 and FcγRIIb1 also have ITIMs in their cytoplasmic domain and function to send inhibitory signals that down regulate or inhibit cell function. It has been shown that these receptors associate with SHP-1 phosphatase via binding to the ITIM motifs. Recruitment of the SHP-1 phosphatase by the receptor appears to be required for intracellular signaling pathways that regulate the inhibitory function of the receptors. Significantly, C-type lectins that are type II membrane proteins having a single intracellular ITIM motif have also been reported. For example, genes localized on human chromosome 12p12-p13 in a region designated as the NK gene complex includes products of the NKG2 complex and CD94, which are involved in recognition of MHC class I molecules and in regulation of NK cell activity. Inhibition of cellular functions by NKG2A/B-CD94 heterdimers is linked to the presence of ITIMs in the NKG2A/B intracellular domain (Lazetic, S. C., et al., J. Immunol. 157:4741, 1996; Houchins, J. P., et al., J. Immunol. 158:3603, 1997).

Thus, by analogy with other C-type lectin family members having ITIM motifs, the polypeptides presented in SEQ ID NO:2, 12 and 16 having ITIM motifs, deliver an inhibitory signal via the interaction of its ITIM with one or more phosphatases, such as tyrosine phosphatases (including SHP-1 tyrosine phosphatase), when the DCL polypeptides are bound with an appropriate receptor or natural ligand. Also by analogy with immunoregulatory receptors possessing ITIMs, DCL family members have a regulatory influence on humoral and cell-mediated immunity, recognition of MHC class I molecules and in regulation of immune cell activity, as well as modulating inflammatory and allergic responses. Clearly, the immune system activatory and inhibitory signals mediated by opposing kinases and phosphatases are very important for maintaining balance in the immune system. Systems with a predominance of activatory signals will lead to autoimmunity and inflammation immune systems with a predominance of inhibitory signals are less able to challenge infected cells or cancer cells. Thus, DCL family members play a role in maintaining balance in the immune system.

Encompassed within the invention are polynucleotides encoding DCL polypeptides. These nucleic acids can be identified in several ways, including isolation of genomic or cDNA molecules from a suitable source. Nucleotide sequences corresponding to the amino acid sequences described herein, to be used as probes or primers for the isolation of nucleic acids or as query sequences for database searches, can be obtained by “back-translation” from the amino acid sequences, or by identification of regions of amino acid identity with polypeptides for which the coding DNA sequence has been identified. The well-known polymerase chain reaction (PCR) procedure can be employed to isolate and amplify a DNA sequence encoding one or more DCL polypeptides or a desired combination of DCL polypeptide fragments. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990).

Polynucleotide or nucleic acid molecules, as used herein, include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention are preferentially derived from human sources, but the invention includes those derived from non-human species, as well.

An “isolated polynucleotide” is a polynucleotide that has been separated from adjacent genetic sequences present in the genome of the organism from which the polynucleotide was isolated, in the case of polynucleotides isolated from naturally occurring sources. In the case of polynucleotides synthesized enzymatically from a template or chemically, such as PCR products, cDNA molecules, or oligonucleotides for example, it is understood that the polynucleotides resulting from such processes are isolated polynucleotides. An isolated polynucleotide molecule may also refer to a polynucleotide molecule in the form of a separate fragment or as a component of a larger polynucleotide construct. In one preferred embodiment, the polynucleotides are substantially free from contaminating endogenous material. The polynucleotide molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

The present invention also includes polynucleotides that hybridize under moderately stringent conditions, and more preferably highly stringent conditions, to polynucleotides encoding DCL polypeptides described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55 degrees C. (or other similar hybridization solutions, such as one containing about 50% formamide, with a hybridization temperature of about 42 degrees C.), and washing conditions of about 60 degrees C., in 0.5×SSC, 0.1% SDS. Generally, highly stringent conditions are defined as hybridization conditions as above, but with washing at approximately 68 degrees C., 0.2×SSC, 0.1% SDS. SSPE (1×SSPE is 0.15M NaCl, 10 mM NaH.sub.2 PO.sub.4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10 degrees C. less than the melting temperature (Tm) of the hybrid, where Tm is determined according to the following equations. For hybrids less than 18 base pairs in length, Tm (degrees C.)=2(# of A+T bases)+4(# of #G+C bases). For hybrids above 18 base pairs in length, Tm (degrees C.)=81.5+16.6(log₁₀ [Na⁺])+0.41(% G+C)−(600/N), where N is the number of bases in the hybrid, and [Na⁺] is the concentration of sodium ions in the hybridization buffer ([Na⁺] for 1×SSC=0.165M). Preferably, each such hybridizing nucleic acid has a length that is at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 85%, at least 90%, at least 95%, at least 97.5%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

Other derivatives of the DCL protein and homologs thereof within the scope of this invention include covalent or aggregative conjugates of the protein or its fragments with other proteins or polypeptides, such as by synthesis in recombinant culture as N-terminal or C-terminal fusions. For example, the conjugated peptide may be a signal (or leader) polypeptide sequence at the N-terminal region of the protein which co-translationally or post-translationally directs transfer of the protein from its site of synthesis to its site of function inside or outside of the cell membrane or wall (e.g., the yeast α-factor leader).

Species homologues (also referred to as an orthologue) of DCL polypeptides and nucleic acids encoding them are also provided by the present invention. As used herein, a “species homologue” is a polypeptide or nucleic acid with a different species of origin from that of a given polypeptide or nucleic acid, but with significant sequence similarity to the given polypeptide or nucleic acid, as determined by those of skill in the art. Species homologues can be isolated and identified by making suitable probes or primers from polynucleotides encoding the amino acid sequences provided herein and screening a suitable nucleic acid source from the desired species. The invention also encompasses allelic variants of DCL polypeptides and nucleic acids encoding them; that is, naturally-occurring alternative forms of such polypeptides and nucleic acids in which differences in amino acid or nucleotide sequence are attributable to genetic polymorphism (allelic variation among individuals within a population).

Protein fusions can comprise peptides added to facilitate purification or identification of DCL proteins and homologs (e.g., poly-His). The amino acid sequence of the inventive proteins can also be linked to an identification peptide such as that described by Hopp et al., Bio/Technology 6:1204 (1988). Such a highly antigenic peptide provides an epitope reversibly bound by a specific monoclonal antibody, enabling rapid assay and facile purification of expressed recombinant protein. The sequence of Hopp et al. is also specifically cleaved by bovine mucosal enterokinase, allowing removal of the peptide from the purified protein. Fusion proteins capped with such peptides may also be resistant to intracellular degradation in E. coli. Fusion proteins further comprise the amino acid sequence of a DCL protein linked to an immunoglobulin Fe region. An exemplary Fc region is a human IgG1 and operative fragments thereof, as well as Fe muteins, which are all well known in the art. Depending on the portion of the Fe region used, a fusion protein may be expressed as a dimer, through formation of interchain disulfide bonds. If the fusion proteins are made with both heavy and light chains of an antibody, it is possible to form a protein oligomer with as many as four DCL regions.

Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

In another embodiment, DCL and homologs thereof further comprise an oligomerizing zipper domain. Zipper domains are well known in the art and need not be described in detail. Examples of leucine zipper domains are those found in the yeast transcription factor GCN4 and a heat-stable DNA-binding protein found in rat liver (C/EBP; Landschulz et al., Science 243:1681, 1989), the nuclear transforming proteins, fos and jun, which preferentially form a heterodimer (O'Shea et al., Science 245:646, 1989; Turner and Tjian, Science 243:1689, 1989), and the gene product of the murine proto-oncogene, c-myc (Landschulz et al., Science 240:1759, 1988). The fusogenic proteins of several different viruses, including paramyxovirus, coronavirus, measles virus and many retroviruses, also possess leucine zipper domains (Buckland and Wild, Nature 338:547, 1989; Britton, Nature 353:394, 1991; Delwart and Mosialos, AIDS Research and Human Retroviruses 6:703, 1990).

The present invention also provides for soluble forms of DCL polypeptides comprising certain fragments or domains of these polypeptides, as previously described above. Soluble DCL polypeptides may be secreted from cells in which they are expressed and preferably retain DCL polypeptide activity. Soluble DCL polypeptides further include oligomers or fusion polypeptides comprising at least one DCL polypeptide, and fragments of any of these polypeptides that have DCL polypeptide activity. A secreted soluble polypeptide can be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble forms of DCL polypeptides is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures.

Derivatives of DCL polypeptides may also be used as immunogens, reagents in in vitro assays, or as binding agents for affinity purification procedures. Such derivatives may also be obtained by cross-linking agents, such as M-maleimidobenzoyl succinimide ester and N-hydroxysuccinimide, at cysteine and lysine residues. The inventive proteins may also be covalently bound through reactive side groups to various insoluble substrates, such as cyanogen bromide-activated, bisoxirane-activated, carbonyldiimidazole-activated or tosyl-activated agarose structures, or by adsorbing to polyolefin surfaces (with or without glutaraldehyde cross-linking). Once bound to a substrate, proteins may be used to selectively bind (for purposes of assay or purification) antibodies raised against the DCL or other proteins which are similar in structure and/or function to the DCL proteins.

The present invention also includes DCL polypeptides with or without associated native-pattern glycosylation. Proteins expressed in yeast or mammalian expression systems, e.g., COS-7 cells, may be similar or slightly different in molecular weight and glycosylation pattern than the native molecules, depending upon the expression system. Expression of DNAs encoding the inventive proteins in bacteria such as E. coli provides non-glycosylated molecules. Functional mutant analogs of DCL protein or homologs thereof having inactivated N-glycosylation sites can be produced by oligonucleotide synthesis and ligation or by site-specific mutagenesis techniques. These analog proteins can be produced in a homogeneous, reduced-carbohydrate form in good yield using yeast expression systems. N-glycosylation sites in eukaryotic proteins are characterized by the amino acid triplet Asn-A₁-Z, where A₁ₗ is any amino acid except Pro, and Z is Ser or Thr. In this sequence, asparagine provides a side chain amino group for covalent attachment of carbohydrate. Such a site can be eliminated by substituting another amino acid for Asn or for residue Z, deleting Asn or Z, or inserting a non-Z amino acid between A₁ and Z, or an amino acid other than Asn between Asn and A₁.

DCL protein derivatives may also be obtained by mutations of the native DCL polypeptide or its subunits. A DCL mutated protein, as referred to herein, is a polypeptide homologous to a DCL protein but which has an amino acid sequence different from the native DCL because of at least one or a plurality of deletions, insertions or substitutions. The effect of any mutation made in a DNA encoding a DCL peptide may be easily determined by analyzing the ability of the mutated DCL peptide to bind proteins that specifically bind DCL (for example, antibodies or natural ligands). Moreover, activity of DCL analogs, muteins or derivatives can be determined by any of the assays methods described herein. Similar mutations may be made in homologs of DCL, and tested in a similar manner.

Bioequivalent analogs of the inventive proteins may be constructed by, for example, making various substitutions of residues or sequences or deleting terminal or internal residues or sequences not needed for biological activity. For example, cysteine residues can be deleted or replaced with other amino acids to prevent formation of incorrect intramolecular disulfide bridges upon renaturation. Other approaches to mutagenesis involve modification of adjacent dibasic amino acid residues to enhance expression in yeast systems in which KEX2 protease activity is present.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., 1998, Acta Phvsiol. Scand. Suppl. 643:55-67; Sasaki et al., 1998, Adv. Biophys. 35:1-24, which discuss alanine scanning mutagenesis).

Desired amino acid substitutions (whether conservative or non-conservative) can be determined by those skilled in the art at the time such substitutions are desired. For example, amino acid substitutions can be used to identify important residues of the peptide sequence, or to increase or decrease the affinity of the peptide or vehicle-peptide molecules (see preceding formulae) described herein. Exemplary amino acid substitutions are set forth in Table 1.

TABLE 1
Amino Acid Substitutions

| Original | Exemplary | Preferred |
|---|---|---|
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, | Leu |
| | Phe, Norleucine | |
| Leu (L) | Norleucine, Ile, Val, | Ile |
| | Met, Ala, Phe | |
| Lys (K) | Arg, 1,4 Diamino- | Arg |
| | butyric Acid, Gln, | |
| | Asn | |
| Met (M) | Leu, Phe, Ile | Leu |
| Phe (F) | Leu, Val, Ile, Ala, | Leu |
| | Tyr | |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, | Leu |
| | Ala, Norleucine | |

In certain embodiments, conservative amino acid substitutions also encompass non-naturally occurring amino acid residues which are typically incorporated by chemical peptide synthesis rather than by synthesis in biological systems.

As noted above, naturally occurring residues may be divided into classes based on common sidechain properties that may be useful for modifications of sequence. For example, non-conservative substitutions may involve the exchange of a member of one of these classes for a member from another class. Such substituted residues may be introduced into regions of the peptide that are homologous with non-human orthologs, or into the non-homologous regions of the molecule. In addition, one may also make modifications using P or G for the purpose of influencing chain orientation.

In making such modifications, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (4.5).

The importance of the hydropathic amino acid index in conferring interactive biological function on a protein is understood in the art. (Kyte, et al., J. Mol. Biol., 157: 105-131 (1982)). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred. One may also identify epitopes from primary amino acid sequences on the basis of hydrophilicity. These regions are also referred to as “epitopic core regions.”

A skilled artisan will be able to determine suitable variants of the polypeptide as set forth in the foregoing sequences using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of a peptide to similar peptides. With such a comparison, one can identify residues and portions of the molecules that are conserved among similar polypeptides. It will be appreciated that changes in areas of a peptide that are not conserved relative to such similar peptides would be less likely to adversely affect the biological activity and/or structure of the peptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the peptide structure.

Additionally, one skilled in the art can review structure-function studies identifying residues in similar peptides that are important for activity or structure. In view of such a comparison, one can predict the importance of amino acid residues in a peptide that correspond to amino acid residues that are important for activity or structure in similar peptides. One skilled in the art may opt for chemically similar amino acid substitutions for such predicted important amino acid residues of the peptides.

One skilled in the art can also analyze the three-dimensional structure and amino acid sequence in relation to that structure in similar polypeptides. In view of that information, one skilled in the art may predict the alignment of amino acid residues of a peptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays know to those skilled in the art. Such data could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See, Moult J., Curr. Op. in Biotech., 7(4): 422427 (1996), Chou et al., Biochemistry, 13(2): 222-245 (1974); Chou et al., Biochemistry, 113(2): 211-222 (1974); Chou et al., Adv. Enzyinol. Relat. Areas Mol. Biol., 47: 45-148 (1978); Chou et al., Ann. Rev. Biochem., 47: 251-276 and Chou et al., Biophys. J., 26: 367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure. See Holm, et al., Nucl. Acid. Res., 27(1): 244-247 (1999). It has been suggested (Brenner et al., Curr. Op. Struct. Biol., 7(3): 369-376 (1997)) that there are a limited number of folds in a given polypeptide or protein and that once a critical number of structures have been resolved, structural prediction will gain dramatically in accuracy.

Additional methods of predicting secondary structure include “threading” (Jones, D., Curr. Opin. Struct. Biol., 7(3): 377-87 (1997); Sippl, et al., Structure, 4(1): 15-9 (1996)), “profile analysis” (Bowie, et al., Science, 253: 164-170 (1991); Gribskov, et al., Meth. Enzym., 183: 146-159 (1990); Gribskov, et al., Proc. Nat. Acad. Sci., 84(13): 4355-8 (1987)), and “evolutionary linkage” (See Holm, supra, and Brenner, supra).

Mutations in nucleotide sequences constructed for expression of analog DCL polypeptides must, of course, preserve the reading frame phase of the coding sequences and preferably will not create complementary regions that could hybridize to produce secondary mRNA structures such as loops or hairpins which would adversely affect translation of the receptor mRNA. Although a mutation site may be predetermined, it is not necessary that the nature of the mutation per se be predetermined. For example, in order to select for optimum characteristics of mutants at a given site, random mutagenesis may be conducted at the target codon and the expressed mutated viral proteins screened for the desired activity.

Not all mutations in the nucleotide sequence that encodes a DCL protein or homolog thereof will be expressed in the final product, for example, nucleotide substitutions may be made to enhance expression, primarily to avoid secondary structure loops in the transcribed mRNA (see EPA 75,444A, incorporated herein by reference), or to provide codons that are more readily translated by the selected host, e.g., the well-known E. coli preference codons for E. coli expression.

Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene having particular codons altered according to the substitution, deletion, or insertion required. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462 disclose suitable techniques, and are incorporated by reference herein.

The DCL polypeptides and analogs described herein will have numerous uses, including the preparation of pharmaceutical compositions. The inventive proteins will also be useful in preparing kits that are used to detect DCL polypeptides, for example, in tissue specimens. Such kits will also find uses in detecting the interaction of DCL polypeptides with their natural ligands, as is necessary when screening for antagonists or mimetics of this interaction (for example, peptides or small molecules that inhibit or mimic, respectively, the interaction). A variety of assay formats are useful in such kits, including (but not limited to) ELISA, dot blot, solid phase binding assays (such as those using a biosensor), rapid format assays and bioassays.

Expression of Recombinant DCL Polypeptides

The polypeptides of the present invention are preferably produced by recombinant DNA methods by inserting a DNA sequence encoding DCL polypeptides or a homolog thereof into a recombinant expression vector and expressing the DNA sequence in a recombinant microbial expression system under conditions promoting expression. DNA sequences encoding the proteins provided by this invention can be assembled from cDNA fragments and short oligonucleotide linkers, or from a series of oligonucleotides, to provide a synthetic gene which is capable of being inserted in a recombinant expression vector and expressed in a recombinant transcriptional unit.

Recombinant expression vectors include synthetic or cDNA-derived DNA fragments encoding DCL polypeptides, homologs, or bioequivalent analogs, operably linked to suitable transcriptional or translational regulatory elements derived from mammalian, microbial, viral or insect genes. Such regulatory elements include a transcriptional promoter, an optional operator sequence to control transcription, a sequence encoding suitable mRNA ribosomal binding sites, and sequences which control the termination of transcription and translation, as described in detail below. The ability to replicate in a host, usually conferred by an origin of replication, and a selection gene to facilitate recognition of transformants may additionally be incorporated.

DNA regions are operably linked when they are functionally related to each other. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; or a ribosome binding site is operably linked to a coding sequence if it is positioned so as to permit translation. Generally, operably linked means contiguous and, in the case of secretory leaders, contiguous and in reading frame. DNA sequences encoding DCL polypeptides or homologs which are to be expressed in a microorganism will preferably contain no introns that could prematurely terminate transcription of DNA into mRNA.

Useful expression vectors for bacterial use can comprise a selectable marker and bacterial origin of replication derived from commercially available plasmids comprising genetic elements of the well-known cloning vector pBR322 (ATCC 37017). Such commercial vectors include, for example, pKK223-3 (Pharmacia Fine Chemicals, Uppsala, Sweden) and pGEM1 (Promega Biotec, Madison, Wis., USA). These pBR322 “backbone” sections are combined with an appropriate promoter and the structural sequence to be expressed. E. coli is typically transformed using derivatives of pBR322, a plasmid derived from an E. coli species (Bolivar et al., Gene 2:95, 1977). pBR322 contains genes for ampicillin and tetracycline resistance and thus provides simple means for identifying transformed cells.

Promoters commonly used in recombinant microbial expression vectors include the β-lactamase (peniicillinase) and lactose promoter system (Chang et al., Nature 275:615, 1978; and Goeddel et al., Nature 281:544, 1979), the tryptophan (trp) promoter system (Goeddel et al., Nucl. Acids Res. 8:4057, 1980; and EPA-36,776) and tac promoter (Maniatis, Molecular Cloning: A Laboratory Manual, Cold Spring Harbor Laboratory, p. 412, 1982). A particularly useful bacterial expression system employs the phage λP_{L} promoter and cI857ts thermolabile repressor. Plasmid vectors available from the American Type Culture Collection which incorporate derivatives of the λP_{L} promoter include plasmid pHUB2, resident in E. coli strain JMB9 (ATCC 37092) and pPLc28, resident in E. coli RR1 (ATCC 53082).

Suitable promoter sequences in yeast vectors include the promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Suitable vectors and promoters for use in yeast expression are further described in R. Hitzeman et al., EPA 73,657.

Preferred yeast vectors can be assembled using DNA sequences from pBR322 for selection and replication in E. coli (Amp^{r} gene and origin of replication) and yeast DNA sequences including a glucose-repressible ADH2 promoter and □-factor secretion leader. The ADH2 promoter has been described by Russell et al. (J. Biol. Chem. 258:2674, 1982) and Beier et al. (Nature 300:724, 1982). The yeast α-factor leader, which directs secretion of heterologous proteins, can be inserted between the promoter and the structural gene to be expressed. See, e.g., Kurjan et al., Cell 30:933, 1982; and Bitter et al., Proc. Natl. Acad. Sci. USA 81:5330, 1984. The leader sequence may be modified to contain, near its 3′ end, one or more useful restriction sites to facilitate fusion of the leader sequence to foreign genes.

The transcriptional and translational control sequences in expression vectors to be used in transforming vertebrate cells may be provided by viral sources. For example, commonly used promoters and enhancers are derived from Polyoma, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin of replication (Fiers et al., Nature 273:113, 1978). Smaller or larger SV40 fragments may also be used, provided the approximately 250 bp sequence extending from the Hind III site toward the BglI site located in the viral origin of replication is included. Further, viral genomic promoter, control and/or signal sequences may be utilized, provided such control sequences are compatible with the host cell chosen. Exemplary vectors can be constructed as disclosed by Okayama and Berg (Mol. Cell. Biol. 3:280, 1983).

A useful system for stable high level expression of mammalian receptor cDNAs in C127 murine mammary epithelial cells can be constructed substantially as described by Cosman et al. (Mol. Immunol. 23:935, 1986). A preferred eukaryotic vector for expression of DCL polynucleotides is referred to as pDC406 (McMahan et al., EMBO J. 10:2821, 1991), and includes regulatory sequences derived from SV40, human immunodeficiency virus (HIV), and Epstein-Barr virus (EBV). Other preferred vectors include pDC409 and pDC410, which are derived from pDC406. pDC410 was derived from pDC406 by substituting the EBV origin of replication with sequences encoding the SV40 large T antigen. pDC409 differs from pDC406 in that a Bgl II restriction site outside of the multiple cloning site has been deleted, making the Bgl II site within the multiple cloning site unique.

A useful cell line that allows for episomal replication of expression vectors, such as pDC406 and pDC409, which contain the EBV origin of replication, is CV-1/EBNA (ATCC CRL 10478). The CV-1/EBNA cell line was derived by transfection of the CV-1 cell line with a gene encoding Epstein-Barr virus nuclear antigen-1 (EBNA-1) and constitutively express EBNA-1 driven from human CMV immediate-early enhancer/promoter.

Host Cells

Transformed host cells are cells which have been transformed or transfected with expression vectors constructed using recombinant DNA techniques and which contain sequences encoding the proteins of the present invention. Transformed host cells may express the desired protein (one or more of the DCL polypeptides or homologs thereof), but host cells transformed for purposes of cloning or amplifying the inventive DNA do not need to express the protein. Expressed proteins will preferably be secreted into the culture supernatant, depending on the DNA selected, but may be deposited in the cell membrane.

Suitable host cells for expression of viral proteins include prokaryotes, eukaryotes, bacterial, yeast, insect, mammalian (human, monkey, ape, rodent, etc.) or other higher order eukaryotic cells under the control of appropriate promoters. Prokaryotes include gram negative or gram positive organisms, for example E. coli or Bacillus spp. Higher eukaryotic cells include established cell lines of mammalian origin as described below. Cell-free translation systems could also be employed to produce viral proteins using RNAs derived from the DNA constructs disclosed herein. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference.

Prokaryotic expression hosts may be used for expression of DCL or homologs that do not require extensive proteolytic and disulfide processing. Prokaryotic expression vectors generally comprise one or more phenotypic selectable markers, for example a gene encoding proteins conferring antibiotic resistance or supplying an autotrophic requirement, and an origin of replication recognized by the host to ensure amplification within the host. Suitable prokaryotic hosts for transformation include E. coli, Bacillus subtilis, Salmonella typhimurium, and various species within the genera Pseudomonas, Streptomyces, and Staphlylococcus, although others may also be employed as a matter of choice.

Recombinant DCL polypeptides may also be expressed in yeast hosts, preferably from the Saccharomyces species, such as S. cerevisiae. Yeast of other genera, such as Pichia or Kluyveromyces may also be employed. Yeast vectors will generally contain an origin of replication from the 2μ yeast plasmid or an autonomously replicating sequence (ARS), promoter, DNA encoding the viral protein, sequences for polyadenylation and transcription termination and a selection gene. Preferably, yeast vectors will include an origin of replication and selectable marker permitting transformation of both yeast and E. coli, e.g., the ampicillin resistance gene of E. coli and S. cerevisiae trp1 gene, which provides a selection marker for a mutant strain of yeast lacking the ability to grow in tryptophan, and a promoter derived from a highly expressed yeast gene to induce transcription of a structural sequence downstream. The presence of the trp1 lesion in the yeast host cell genome then provides an effective environment for detecting transformation by growth in the absence of tryptophan.

Suitable yeast transformation protocols are known to those of skill in the art; an exemplary technique is described by Hinnen et al., Proc. Natl. Acad. Sci. USA 75:1929, 1978, selecting for Trp⁺ transformants in a selective medium consisting of 0.67% yeast nitrogen base, 0.5% casamino acids, 2% glucose, 10 μg/ml adenine and 20 μg/ml uracil. Host strains transformed by vectors comprising the ADH2 promoter may be grown for expression in a rich medium consisting of 1% yeast extract, 2% peptone, and 1% glucose supplemented with 80 μg/ml adenine and 80 μg/ml uracil. Derepression of the ADH2 promoter occurs upon exhaustion of medium glucose. Crude yeast supernatants are harvested by filtration and held at 4° C. prior to further purification.

The inventive polypeptide can also be produced by operably linking the isolated nucleic acid of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac®) kit), and such methods are well known in the art, as described in Summers and Smith, Texas Agricultural Experiment Station Bulletin No. 1555 (1987), and Luckow and Summers, Bio/Technology 6:47 (1988). Cell-free translation systems could also be employed to produce polypeptides using RNAs derived from nucleic acid constructs disclosed herein.

Various mammalian or insect cell culture systems can be employed to express recombinant protein. Baculovirus systems for production of heterologous proteins in insect cells are reviewed by Luckow and Summers, Bio/Technology 6:47. (1988). Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells or their derivatives such as Veggie CHO and related cell lines which grow in serum-free media (Rasmussen et al., 1998, Cytotechnology 28: 31), HeLa cells, BHK (ATCC CRL 10) cell lines, the CV1/EBNA cell line derived from the African green monkey kidney cell line CV1 (ATCC CCL 70) (McMahan et al., 1991, EMBO J. 10: 2821, 1991), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays. Mammalian expression vectors may comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences.

Purification of DCL Polypeptides

The polypeptides may also be isolated and purified in accordance with conventional methods of recombinant synthesis. For example, a lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Purified DCL polypeptides, variants, homologs, or analogs are prepared by culturing suitable host/vector systems to express the recombinant translation products of the DNAs of the present invention, which are then purified from culture media or cell extracts. For example, supernatants from systems which secrete recombinant protein into culture media can be first concentrated using a commercially available protein concentration filter, for example, an Amicon or Millipore Pellicon ultrafiltration unit.

Following the concentration step, the concentrate can be applied to a suitable purification matrix. For example, a suitable affinity matrix can comprise a counter structure protein or antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Gel filtration chromatography also provides a means of purifying the inventive proteins.

Affinity chromatography is a particularly preferred method of purifying DCL polypeptides and variants, homologs, or analogs thereof. For example, a DCL polypeptide expressed as a fusion protein comprising an immunoglobulin Fc region can be purified using Protein A or Protein G affinity chromatography. Moreover, a DCL protein comprising an oligomerizing zipper domain may be purified on a resin comprising an antibody specific to the oligomerizing zipper domain. Monoclonal antibodies against the DCL protein may also be useful in affinity chromatography purification, by utilizing methods that are well-known in the art. A ligand, such as a carbohydrate or glycolprotein moiety may also be used to prepare an affinity matrix for affinity purification of DCL.

Finally, one or more reversed-phase high performance liquid chromatography (RP-HPLC) steps employing hydrophobic RP-HPLC media, e.g., silica gel having pendant methyl or other aliphatic groups, can be employed to further purify a DCL composition. Some or all of the foregoing purification steps, in various combinations, can also be employed to provide a homogeneous recombinant protein.

Recombinant protein produced in bacterial culture is usually isolated by initial extraction from cell pellets, followed by one or more concentration, salting-out, aqueous ion exchange or size exclusion chromatography steps. Finally, high performance liquid chromatography (HPLC) can be employed for final purification steps. Microbial cells employed in expression of recombinant viral protein can be disrupted by any convenient method, including freeze-thaw cycling, sonication, mechanical disruption, or use of cell lysing agents.

Fermentation of yeast that express the inventive protein as a secreted protein greatly simplifies purification. Secreted recombinant protein resulting from a large-scale fermentation can be purified by methods analogous to those disclosed by Urdal et al. (J. Chromatog. 296:171, 1984). This reference describes two sequential, reversed-phase HPLC steps for purification of recombinant human GM-CSF on a preparative HPLC column.

Protein synthesized in recombinant culture is characterized by the presence of cell components, including proteins, in amounts and of a character which depend upon the purification steps taken to recover the inventive protein from the culture. These components ordinarily will be of yeast, prokaryotic or non-human higher eukaryotic origin and preferably are present in innocuous contaminant quantities, on the order of less than about 1 percent by weight. Further, recombinant cell culture enables the production of the inventive proteins free of other proteins which may be normally associated with the proteins as they are found in nature in the species of origin.

Screening Assays and Methods

The present invention provides methods for screening for a molecule (often referred to as a “test compound”) that antagonizes or agonizes the activity of DCL polypeptides and DCL-associated substrates and/or binding partners. DCL polypeptide activities include, but are not limited to, antigen binding, internalization, processing and presentation; APC activation, differentiation, maturation, homing and transmigration; cell to cell interactions including binding and modulation of intracellular signaling pathways in either an excitatory or inhibitory manner, as well as extracellular communication through pathways leading to secretion of factors that act in an autocrine, paracrine and/or endocrine fashion. Examples of cells that may bind to APCs expressing DCL polypeptides include cells of the immune system, including DCs, T-cells, B-cells, NK cells, as well as precursors thereof.

Binding partner, as used herein, may comprise a natural ligand, which may be an/a oligosaccharide, polysaccharide, carbohydrate, glycoprotein, phospholipid, glycolipid, glycosphingolipid and the like; preferably, the natural ligand is selected from the group consisting of bacterial, viral, fungal or protazoan polypeptides, as well as cell membrane-associated polypeptides. A binding partner may also comprise an antibody, either agonistic or antagonistic to DCL activity. Also, a binding partner may comprise a fragment, derivative, fusion protein or peptidomimetic of a DCL natural ligand.

In the most basic sense, illustrative assays comprise a method for identifying test compounds that modulate DCL polypeptide activity, which may be in the form of agonist or antagonists, comprising mixing a test compound with one or more DCL polypeptides and determining whether the test compound alters the DCL polypeptide activity of said polypeptide. Other embodiments comprise a method for identifying compounds that inhibit the binding activity of DCL polypeptides comprising mixing a test compound with one or more DCL polypeptides and a binding partner of said polypeptide and determining whether the test compound inhibits the binding activity of said polypeptide.

Additional embodiments include methods of screening for active compounds with particularized biological readouts, such as for example, modulating C-type lectin activity. As used throughout this application, modulate means to either increase or decrease activity. Further embodiments may use modulation of aspartyl protease activity as a biological readout. And, in further embodiments biological readouts may include modulating ITIM activity (as well as associated pathways, such as interactions with ITAM domains and one or more phosphatases, such as tyrosine phosphatases including. SHP-1 tyrosine phosphatase).

The methods of the invention may be used to identify antagonists and agonists of DCL signaling activity from cells, cell-free preparations, chemical libraries, cDNA libraries, recombinant antibody libraries (or libraries comprising subunits of antibodies) and natural product mixtures. The antagonists and agonists may be natural or modified substrates, ligands, enzymes, receptors, etc. of the polypeptides of the instant invention, or may be structural or functional mimetics of one of the DCL polypeptides and fragments thereof. Potential antagonists of the instant invention may include small molecules, peptides and antibodies that bind to and occupy a binding site of the inventive polypeptides or a binding partner thereof, causing them to be unavailable to bind to their natural binding partners and therefore preventing normal biological activity. Potential agonists include small molecules, peptides and antibodies which bind to the instant polypeptides or binding partners thereof, and elicit the same or enhanced biologic effects as those caused by the binding of the polypeptides of the instant invention.

In one aspect, the inventive methods utilize homogeneous assay formats such as fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence. In another aspect, the inventive methods utilize heterogeneous assay formats such as enzyme-linked immunosorbant assays (EUSA) or radioimmunoassays. In yet another aspect of the invention are cell-based assays, for example those utilizing reporter genes, as well as functional assays that analyze the effect of an antagonist or agonist on biological function(s).

Small molecule agonists and antagonists are usually less than 10K molecular weight and may possess a number of physicochemical and pharmacological properties which enhance cell penetration, resist degradation and prolong their physiological half-lives (Gibbs, J., Pharmaceutical Research in Molecular Oncology, Cell, Vol. 79 (1994)). Antibodies, which include intact molecules as well as fragments such as Fab and F(ab′)₂ fragments, as well as recombinant molecules derived therefrom (including antibodies expressed on phage, intrabodies, single chain antibodies such as scFv and other molecules derived from immunoglobulins that are known in the art), may be used to bind to and inhibit the polypeptides of the instant invention by blocking the propagation of a signaling cascade. It is preferable that the antibodies are humanized, and more preferable that the antibodies are human. The antibodies of the present invention may be prepared by any of a variety of well-known methods.

Additional examples of candidate molecules, also referred to herein as “test compounds,” to be tested for DCL agonist or antagonist activity include, but are not limited to, carbohydrates, small molecules (usually organic molecules or peptides), proteins, and nucleic acid molecules (including oligonucleotide fragments typically consisting of from 8 to 30 nucleic acid residues). Peptides to be tested typically consist of from 5 to 25 amino acid residues. Also, candidate nucleic acid molecules can be antisense nucleic acid sequences, and/or can possess ribozyme activity. Candidate molecules that can be assayed for DCL agonist or antagonist activity may also include, but are not limited to, small organic molecules, such as those that are commercially available—often as part of large combinatorial chemistry compound ‘libraries’—from companies such as Sigma-Aldrich (St. Louis, Mo.), Arqule (Woburn, Mass.), Enzymed (Iowa City, Iowa), Maybridge Chemical Co. (Trevillett, Cornwall, UK), MDS Panlabs (Bothell, Wash.), Pharmacopeia (Princeton, N.J.), and Trega (San Diego, Calif.). Compounds including natural products, inorganic chemicals, and biologically active materials such as proteins and toxins can also be assayed using these methods for the ability to modulate DCL-associated cellular events.

Specific screening methods are known in the art and along with integrated robotic systems and collections of chemical compounds/natural products are extensively incorporated in high throughput screening so that large numbers of test compounds can be tested for antagonist or agonist activity within a short amount of time. These methods include homogeneous assay formats such as fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence, as well as more traditional heterogeneous assay formats such as enzyme-linked immunosorbant assays (ELISA) or radioimmunoassays. Homogeneous assays are preferred. Also comprehended herein are cell-based assays, for example those utilizing reporter genes, as well as functional assays that analyze the effect of an antagonist or agonist on biological function(s) (for example, phosphorylation of substrates, secretion of cytokines or growth factors, proliferation and/or differentiation of cells or tissues, and the like).

Moreover, combinations of screening assays can be used to find molecules that regulate the biological activity of DCL. Molecules that regulate the biological activity of a polypeptide may be useful as agonists or antagonists of the peptide. In using combinations of various assays, it is usually first determined whether a candidate molecule binds to a polypeptide by using an assay that is amenable to high throughput screening. Binding candidate molecules identified in this manner are then added to a biological assay to determine biological effects. Molecules that bind and that have an agonistic or antagonistic effect on biologic activity will be useful in treating or preventing disease or conditions with which the polypeptide(s) are implicated.

Generally, an antagonist will inhibit the activity by at least 30%; more preferably, antagonists will inhibit activity by at least 50%, most preferably by at least 90%. Similarly, an agonist will enhance the activity by at least 20%; more preferably, agonists will enhance activity by at least 30%, most preferably by at least 50%. Those of skill in the art will recognize that agonists and/or antagonists with different levels of agonism or antagonism respectively may be useful for different applications (i.e., for treatment of different disease states).

Homogeneous assays are mix-and-read style assays that are very amenable to robotic application, whereas heterogeneous assays require separation of free from bound analyte by more complex unit operations such as filtration, centrifugation or washing. These assays are utilized to detect a wide variety of specific biomolecular interactions (including protein-protein, receptor-ligand, enzyme-substrate, and so on), and the inhibition thereof by small organic molecules. These assay methods and techniques are well known in the art (see, e.g., High Throughput Screening: The Discovery of Bioactive Substances, John P. Devlin (ed.), Marcel Dekker, New York, 1997 ISBN: 0-8247-0067-8). The screening assays of the present invention are amenable to high throughput screening of chemical libraries and are suitable for the identification of small molecule drug candidates, antibodies, peptides, and other antagonists and/or agonists, natural or synthetic.

One such assay is based on fluorescence resonance energy transfer (FRET; for example, HTRF®, Packard BioScience Company, Meriden, Conn.; LANCE™, PerkinElmer LifeSciences, Wallac Oy., Turku, Finland) between two fluorescent labels, an energy donating long-lived chelate label and a short-lived organic acceptor. The energy transfer occurs when the two labels are brought in close proximity via the molecular interaction between DCL and a substrate and/or binding partner. In a FRET assay for detecting inhibition of the binding of DCL and a substrate and/or binding partner, europium chelate or cryptate labeled DCL or substrate and/or binding partner serves as an energy donor and streptavidin-labeled allophycocyanin (APC) bound to the appropriate binding partner (i.e., substrate and/or binding partner if DCL is labeled, or DCL if a substrate or binding partner is labeled) serves as an energy acceptor. Once DCL associates with a substrate and/or binding partner, the donor and acceptor molecules are brought in close proximity, and energy transfer occurs, generating a fluorescent signal at 665 nm. Antagonists of the interaction of DCL and a substrate and/or binding partner will thus inhibit the fluorescent signal, whereas agonists of this interaction would enhance it.

Another useful assay is a bioluminescence resonance energy transfer, or BRET, assay, substantially as described in Xu et al., Proc. Natl. Acad. Sci. USA 96:151 (1999). Similar to a FRET assay, BRET is based on energy transfer from a bioluminescent donor to a fluorescent acceptor protein. However, a green fluorescent protein (GFP) is used as the acceptor molecule, eliminating the need for an excitation light source. Exemplary BRET assays include BRET and BRET² from Packard BioScience, Meriden, Conn.

DELFIA® (dissociated enhanced lanthanide fluoroimmunoassay; PerkinElmer LifeSciences, Wallac Oy., Turku, Finland) is a solid-phase assay based on time-resolved fluorometry analysis of lanthanide chelates (see, for example, U.S. Pat. No. 4,565,790, issued Jan. 21, 1986). For this type of assay, microwell plates are coated with a first protein (NEMO or CYLD). The binding partner (DCL or a substrate and/or binding partner of DCL, respectively) is conjugated to europium chelate or cryptate, and added to the plates. After suitable incubation, the plates are washed and a solution that dissociates europium ions from solid phase bound protein, into solution, to form highly fluorescent chelates with ligands present in the solution, after which the plates are read using a reader such as a VICTOR²™ (PerkinElmer LifeSciences, Wallac Oy., Turku, Finland) plate reader to detect emission at 615 nm).

Another assay that may be useful in the inventive methods is a FlashPlate® (Packard Instrument Company, IL)-based assay. This assay measures the ability of compounds to inhibit protein-protein interactions. FlashPlates® are coated with a first protein (either DCL or a substrate and/or binding partner of DCL), then washed to remove excess protein. For the assay, compounds to be tested are incubated with the second protein (a substrate and/or binding partner of DCL, if the plates are coated with DCL, or DCL if plates are coated with a substrate and/or binding partner of DCL) and 1125 labeled antibody against the second protein and added to the plates. After suitable incubation and washing, the amount of radioactivity bound is measured using a scintillation counter (such as a MicroBeta® counter; PerkinElmer LfeSciences, Wallac Oy., Turku, Finland).

The AlphaScreen™ assay (Packard Instrument Company, Meriden, Conn.). AlphaScreen™ technology is an “Amplified Luminescent Proximity Homogeneous Assay” method utilizing latex microbeads (250 nm diameter) containing a photosensitizer (donor beads), or chemiluminescent groups and fluorescent acceptor molecules (acceptor beads). Upon illumination with laser light at 680 nm, the photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity to the donor bead (i.e., by virtue of the interaction of DCL and a substrate and/or binding partner of DCL), the singlet-state oxygen molecules reacts with chemiluminescent groups in the acceptor beads, which immediately transfer energy to fluorescent acceptors in the same bead. These fluorescent acceptors shift the emission wavelength to 520-620 nm, resulting in a detectable signal. Antagonists of the interaction of of DCL and a substrate and/or binding partner of DCL will thus inhibit the shift in emission wavelength, whereas agonists of this interaction would enhance it.

Polypeptides of the DCL family and fragments thereof can be used to identify binding partners. For example, they can be tested for the ability to bind a candidate binding partner in any suitable assay, such as a conventional binding assay, as well as a yeast two hybrid system. To illustrate, the DCL polypeptide can be labeled with a detectable reagent (e.g., a radionuclide, chromophore, enzyme that catalyzes a calorimetric or fluorometric reaction, and the like). The labeled polypeptide is contacted with cells expressing the candidate binding partner. The cells then are washed to remove unbound labeled polypeptide, and the presence of cell-bound label is determined by a suitable technique, chosen according to the nature of the label.

One example of a binding assay procedure is as follows. A recombinant expression vector containing the candidate binding partner cDNA is constructed. CV1-EBNA-1 cells in 10 cm² dishes are transfected with this recombinant expression vector. CV-1/EBNA-1 cells (ATCC CRL 10478) constitutively express EBV nuclear antigen-1 driven from the CMV Immediate-early enhancer/promoter. CV1-EBNA-1 was derived from the African Green Monkey kidney cell line CV-1 (ATCC CCL 70), as described by McMahan et al., (EMBO J. 10:2821, 1991). The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated for 1 hour at 37° C. with various concentrations of, for example, a soluble polypeptide/Fc fusion polypeptide made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization. The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc polypeptide that has bound to the cells. In all assays, non-specific binding of ¹²⁵I-antibody is assayed in the absence of the Pc fusion polypeptide/Fc, as well as in the presence of the Fc fusion polypeptide and a 200-fold molar excess of unlabeled mouse anti-human IgG antibody. Cell-bound ¹²⁵I-antibody is quantified on a Packard Autogamma counter. Affinity calculations (Scatchard, Ann. N.Y. Acad. Sci. 51:660, 1949) are generated on RS/1 (BBN Software, Boston, Mass.) run on a Microvax computer. Binding can also be detected using methods that are well suited for high-throughput screening procedures, such as scintillation proximity assays (Udenfriend et al., 1985, Proc Natl Acad Sci USA 82: 8672-8676), homogeneous time-resolved fluorescence methods (Park et al., 1999, Anal Biochem 269: 94-104), fluorescence resonance energy transfer (FRET) methods (Clegg R M, 1995, Curr Opin Biotechnol 6: 103-110), or methods that measure any changes in surface plasmon resonance when a bound polypeptide is exposed to a potential binding partner, using for example a biosensor such as that supplied by Biacore AB (Uppsala, Sweden).

Yeast Two-Hybrid or “Interaction Trap” assays may be used in screening for test compounds. Where the DCL polypeptide binds or potentially binds to another polypeptide, the nucleic acid encoding the DCL polypeptide can also be used in interaction trap assays (such as, for example, that described in Gyuris et al., Cell 75:791-803 (1993)) to identify nucleic acids encoding the other polypeptide with which binding occurs or to identify inhibitors of the binding interaction. Polypeptides involved in these binding interactions can also be used to screen for peptide or small molecule inhibitors or agonists of the binding interaction.

Another type of suitable binding assay is a competitive binding assay. To illustrate, biological activity of a variant can be determined by assaying for the variant's ability to compete with the native polypeptide for binding to the candidate binding partner. Competitive binding assays can be performed by conventional methodology. Reagents that can be employed in competitive binding assays include radiolabeled DCL and intact cells expressing DCL (endogenous or recombinant) on the cell surface. For example, a radiolabeled soluble DCL fragment can be used to compete with a soluble DCL variant for binding to cell surface receptors. Instead of intact cells, one could substitute a soluble binding partner/Fc fusion polypeptide bound to a solid phase through the interaction of Polypeptide A or Polypeptide G (on the solid phase) with the Fc moiety. Chromatography columns that contain Polypeptide A and Polypeptide G include those available from Pharmacia Biotech, Inc., Piscataway, N.J.

Cell proliferation, cell death, cell differentiation and cell adhesion assays may also be used to screen for test compounds. A DCL polypeptide, fragment and/or derivative thereof of the present invention may exhibit cytoline, cell proliferation (either inducing or inhibiting), or cell differentiation (either inducing or inhibiting) activity, or may induce production of other cytolines, chemokines or other soluble factor in certain cell populations. Many polypeptide factors discovered to date have exhibited such activity in one or more factor-dependent cell proliferation assays, and hence the assays serve as a convenient confirmation of cell stimulatory activity. The activity of agonists and/or antagonists of DCL of the present invention is evidenced by any one of a number of routine factor-dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK. The activity of a DCL polypeptide of the invention may, among other means, be measured by the following methods:

Assays for cytokine production and/or proliferation of spleen cells, lymph node cells or thymocytes include, without limitation, those described in: Kruisbeek and Shevach, 1994, Polyclonal T cell stimulation, in Current Protocols in Immunology, Coligan et al. eds. Vol 1 pp. 3.12.1-3.12.14, John Wiley and Sons, Toronto; and Schreiber, 1994, Measurement of mouse and human interferon gamma in Current Protocols in Immunology, Coligan et al. eds. Vol 1 pp. 6.8.1-6.8.8, John Wiley and Sons, Toronto.

Assays for cell movement and adhesion include, without limitation, those described in: Current Protocols in Immunology Coligan et al. eds, Greene Publishing Associates and Wiley-Interscience (Chapter 6.12, Measurement of alpha and beta chemokines 6.12.1-6.12.28); Taub et al. J. Clin. Invest. 95:1370-1376, 1995; Lind et al. APMIS 103:140-146, 1995; Muller et al Eur. J. Immunol. 25: 1744-1748; Gruber et al. J. Immunol. 152:5860-5867, 1994; Johnston et al. J Immunol. 153: 1762-1768, 1994 Assays for receptor-ligand activity include without limitation those described in: Current Protocols in Immunology Coligan et al. eds, Greene Publishing Associates and Wiley-Interscience (Chapter 7.28, Measurement of cellular adhesion under static conditions 7.28.1-7.28.22), Takai et al., Proc. Natl. Acad. Sci. USA 84:6864-6868, 1987; Bierer et al., J. Exp. Med. 168:1145-1156,1988; Rosenstein et al., J. Exp. Med. 169:149-160 1989; Stoltenborg et al., J. Immunol. Methods 175:59-68, 1994; Stitt et al., Cell 80:661-670, 1995.

Methods of the present invention may be used to screen for antisense molecules that inhibit the functional expression of one or more mRNA molecules that encode one or more proteins that mediate a DCL-dependent cellular response. An anti-sense nucleic acid molecule is a DNA sequence that is inverted relative to its normal orientation for transcription and so expresses an RNA transcript that is complementary to a target mRNA molecule expressed within the host cell (i.e., the RNA transcript of the anti-sense nucleic acid molecule can hybridize to the target mRNA molecule through Watson-Crick base pairing). An anti-sense nucleic acid molecule may be constructed in a number of different ways provided that it is capable of interfering with the expression of a target protein. Typical anti-sense oligonucleotides to be screened preferably are 30-40 nucleotides in length. The anti-sense nucleic acid molecule generally will be substantially identical (although in antisense orientation) to the target gene. The minimal identity will typically be greater than about 65%, but a higher identity might exert a more effective repression of expression of the endogenous sequences. Substantially greater identity of more than about 80% is preferred, though about 95% to absolute identity would be most preferred.

Candidate nucleic acid molecules may possess ribozyme activity. Thus, the methods of the invention can be used to screen for ribozyme molecules that inhibit the functional expression of one or more mRNA molecules that encode one or more proteins that mediate a CD40 dependent cellular response. Ribozymes are catalytic RNA molecules that can cleave nucleic acid molecules having a sequence that is completely or partially homologous to the sequence of the ribozyme. It is possible to design ribozyme transgenes that encode RNA ribozymes that specifically pair with a target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs. The design and use of target RNA-specific ribozymes is described in Haseloff et al. (Nature, 334:585, 1988; see also U.S. Pat. No. 5,646,023), both of which publications are incorporated herein by reference. Tabler et al. (Gene 108:175, 1991) have greatly simplified the construction of catalytic RNAs by combining the advantages of the anti-sense RNA and the ribozyme technologies in a single construct. Smaller regions of homology are required for ribozyme catalysis, therefore this can promote the repression of different members of a large gene family if the cleavage sites are conserved.

Rational Drug Design

The goal of rational drug design is to produce structural analogs of biologically active polypeptides of interest or of small molecules with which they interact, e.g., inhibitors, agonists, antagonists, etc. Any of these examples can be used to fashion drugs which are more active or stable forms of the active compound or which enhance or interfere with the function of a DCL active compound in vivo (Hodgson J (1991) Biotechnology 9:19-21). In one approach, the three-dimensional structure of an active compound of interest is determined by x-ray crystallography, by nuclear magnetic resonance, or by computer homology modeling or, most typically, by a combination of these approaches. Both the shape and charges of the active compound must be ascertained to elucidate the structure and to determine active site(s) of the molecule. Less often, useful information regarding the structure of a polypeptide may be gained by modeling based on the structure of homologous polypeptides. In both cases, relevant structural information is used to design analogous DCL-like molecules, to identify efficient inhibitors, or to identify small molecules that bind DCL polypeptides or DCL-associated substrates and/or binding partners. Useful examples of rational drug design include molecules which have improved activity or stability as shown by Braxton S and Wells J A (1992 Biochemistry 31:7796-7801) or which act as inhibitors, agonists, or antagonists of native peptides as shown by Athauda S B et al (1993 J Biochem 113:742-746). The use of DCL polypeptide structural information in molecular modeling software systems to assist in agonists and/or antagonist design and in studying agonists/antagonists-DCL polypeptide interaction is also encompassed by the invention. A particular method of the invention comprises analyzing the three dimensional structure of DCL polypeptides for likely binding sites of substrates, synthesizing a new molecule that incorporates a predictive reactive site, and assaying the new molecule as described further herein.

It is also possible to isolate a target-specific antibody, selected by functional assay, as described further herein, and then to solve its crystal structure. This approach, in principle, yields a pharmacore upon which subsequent drug design can be based. It is possible to bypass polypeptide crystallography altogether by generating anti-idiotypic antibodies (anti-ids) to a functional, pharmacologically active antibody. As a mirror image of a mirror image, the binding site of the anti-ids would be expected to be an analog of the original antigen. The anti-id could then be used to identify and isolate peptides from banks of chemically or biologically produced peptides. The isolated peptides would then act as the pharmacore.

The purified DCL polypeptides of the invention (including polypeptides and fragments thereof, muteins, variants, oligomers, fusion proteins, and other forms) are useful in a variety of assays. For example, the DCL molecules of the present invention can be used to identify binding partners of DCL polypeptides, which can also be used to modulate intercellular communication, cell stimulation, or immune cell activity. Alternatively, they can be used to identify non-binding-partner molecules or substances that modulate intercellular communication, cell stimulatory pathways, or immune cell activity.

Therapeutic Applications

Methods provided herein comprise administering DCL polypeptides and/or agonists and/or antagonists thereof to a patient, thereby modulating biological responses mediated by DCL proteins on antigen presenting cells, which in turn play a role in a particular condition. DCL polypeptide activities that may play a role in a particular condition include, but are not limited to, antigen binding, internalization, processing and presentation; APC activation, differentiation, maturation, homing and transmigration; cell to cell interactions including binding and modulation of intracellular signaling pathways in either an excitatory or inhibitory manner, as well as extracellular communication through pathways leading to secretion of factors that act in an autocrine, paracrine and/or endocrine fashion. Examples of cells that may bind to APCs expressing DCL polypeptides include cells of the immune system, including DCs, T-cells, B-cells, NK cells, as well as precursors thereof.

Treatment encompasses alleviation of at least one symptom of a disorder, or reduction of disease severity, and the like. An antagonist need not effect a complete “cure”, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents.

Polynucleotides and polypeptides of the present invention may be used to treat or prevent disease states associated with infectious agents, as well as augment an immune response to infectious agents. In one embodiment, bacterial and/or viral antigens are targeted to APCs-preferably DCs, that express DCL polypeptides. The present invention provides compositions for targeting bacterial and/or viral antigens to APCs. For example, one or more DCL polypeptide agonists, are bound or chemically linked or coupled with one or more bacterial or viral antigens and administered in vivo, or by established ex vivo methods, to a patient in need thereof in order to facilitate antigen uptake and presentation in APCs expressing DCL polypeptides. Examples of DCL agonists include, for example, anti-DCL antibodies, or derivative thereof, a DCL natural ligand, or derivatives and peptide mimetics thereof, as well as anti-idiotypic antibodies directed against anti-natural ligand antibodies.

The present invention also provides methods for treating or preventing disease states associated with infectious agents, as well as augmenting an immune response to infectious agents, the method comprising administering to a patient in need thereof one or more DCL agonists that have been bound to or chemically coupled or linked to one or more bacterial or viral antigens.

In alternative embodiments, the present invention provides methods of augmenting an immune response to infectious agents, the method comprising administering to a patient in need thereof one or more DCL agonists that have been bound to or chemically coupled or linked to one or more bacterial or viral antigens wherein the DCL polypeptides also facilitate trafficking to peripheral lymph nodes for antigen presentation to T and B cells located therein. In additional embodiments, DCL agonists may be used to alter the pattern of APC trafficking to specific organs of choice, such as preferentially trafficking to draining lymph nodes, spleen and the like.

In yet another embodiment, the present invention provides methods of augmenting an immune response to infectious agents, the method comprising administering to a patient in need thereof one or more DCL agonists that have been bound to or chemically coupled or linked to one or more bacterial or viral antigens wherein the DCL polypeptides also facilitate trafficking to peripheral lymph nodes for antigen presentation to T cells, and wherein the DCL polypeptides also facilitate binding to and costimulation of T cells.

Examples of infectious virus include: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunga viruses, phleboviruses and Nairo viruses); Arena viridae (hemorrhagic fever viruses); Reoviridae (e.g., reoviruses, orbiviuises and rotaviruses); Birnaviridae; Hepadnaviridae (Hepatitis B virus); Parvoviridae (parvovirusies); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses'); Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of infectious bacteria include: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

Polynucleotides and polypeptides of the present invention may be used to treat disease states associated with fungal infections or parasitic infestations, as well as augment an immune response to those disorders. In one embodiment, fungal or parasitic antigens are targeted to APCs, preferably DCs expressing DCL polypeptides. Therefore, the present invention provides compositions for targeting fungal or parasitic antigens to APCs. One or more DCL polypeptides agonists, are bound or chemically linked or coupled with one or more fungal or parasitic antigens and administered either in vivo or by established ex vivo methods to a patient in need thereof in order to facilitate antigen uptake and presentation in APCs expressing DCL polypeptides. Examples of DCL agonists include, for example, an anti-DCL antibodies, or derivative thereof, a DCL natural ligand, or derivatives and peptide mimetic thereof and anti-idiotypic antibodies directed against anti-natural ligand antibodies.

The present invention also provides methods for treating disease states associated with fungal infections or parasitic infestations, as well as augmenting an immune response to fungal infections or parasitic infestations, the method comprising administering to a patient in need thereof one or more DCL agonists that have been bound to or chemically coupled or linked to one or more fungal or parasitic antigens.

In alternative embodiments, the present invention provides methods of augmenting an immune response to fungal infections or parasitic infestations, the method comprising administering to a patient in need thereof one or more DCL agonists that have been bound to or chemically coupled or linked to one or more fungal or parasitic antigens wherein the DCL polypeptides also facilitate trafficking to peripheral lymph nodes for antigen presentation to T and B cells located therein.

In yet another embodiment, the present invention provides methods of augmenting an immune response to fungal infections or parasitic infestations, the method comprising administering to a patient in need thereof one or more DCL agonists that have been bound to or chemically coupled or linked to one or more fungal or parasitic antigens wherein the DCL polypeptides also facilitate trafficking to peripheral lymph nodes for antigen presentation to T cells, and wherein the DCL polypeptides also facilitate binding to and costimulation of T cells.

Examples of infectious organisms may include, but is not limited to, Cryptococcus neoformans, Histoplasma capsulatum, Coccidiodes immitis, Blastomyces dennatitidis, Chlamydia trachomatis, Candida albicans and the like. Examples of infectious organisms include Plasmodium falciparum and Toxoplasma gondii.

Polynucleotides and polypeptides of the present invention may be used to treat disease states associated with various hematologic and oncologic disorders, as well as augment an immune response to those disorders. In one embodiment, tumor antigens are targeted to APCs, preferably DCs expressing DCL polypeptides. Therefore, the present invention provides compositions for targeting tumor antigens to APCs. One or more DCL polypeptides agonists, are bound or chemically linked or coupled with one or more tumor antigens and administered either in vivo or by established ex vivo methods to a patient in need thereof in order to facilitate antigen uptake and presentation in APCs expressing DCL polypeptides. Examples of DCL agonists include, for example, an anti-DCL antibodies, or derivative thereof, a DCL natural ligand, or derivatives and peptide mimetic thereof and anti-idiotypic antibodies directed against anti-natural ligand antibodies.

The present invention also provides methods for treating disease states associated with cancer, hematologic and oncologic disorders, as well as augmenting an immune response to hematologic and oncologic disorders, the method comprising administering to a patient in need thereof one or more DCL agonists that have been bound to or chemically coupled or linked to one or more tumor antigens.

In alternative embodiments, the present invention provides methods of augmenting an immune response to hematologic and oncologic disorders, the method comprising administering to a patient in need thereof one or more DCL agonists that have been bound to or chemically coupled or linked to one or more bacterial or viral antigens wherein the DCL polypeptides also facilitate trafficking to peripheral lymph nodes for antigen presentation to T and B cells located therein.

In yet another embodiment, the present invention provides methods of augmenting an immune response to hematologic and oncologic disorders, the method comprising administering to a patient in need thereof one or more DCL agonists that have been bound to or chemically coupled or linked to one or more bacterial or viral antigens wherein the DCL polypeptides also facilitate trafficking to peripheral lymph nodes for antigen presentation to T cells, and wherein the DCL polypeptides also facilitate binding to and costimulation of T cells.

Tumor antigens are well known in the art, such as those described in Minev, B., et al., Pharmacol. Ther., Vol. 81, No. 2, pp. 121-139, 1999, and may also include tumor antigens associated with the following examples. Tumor antigens may be isolated, i.e., partially purified, cell-associated or some form of fusion protein.

Examples of hematologic and oncologic disorders include acute myelogenous leukemia, Epstein-Barr virus-positive nasopharyngeal carcinoma, glioma, colon, stomach, prostate, renal cell, cervical and ovarian cancers, lung cancer (SCLC and NSCLC), including cancer-associated cachexia, fatigue, asthenia, paraneoplastic syndrome of cachexia and hypercalcemia. In addition, solid tumors, including sarcoma, osteosarcoma, and carcinoma, such as adenocarcinoma (for example, breast cancer); melanotic neoplasia, including melanocytic nevus, radial and vertical growth phase melanoma; squamous cell neoplasia, including seborrheic keratosis, actinic keratosis, basal cell carcinomas and squamous cell carcinoma. Furthermore, leukemia, including acute myelogenous leukemia, chronic or acute lymphoblastic leukemia and hairy cell leukemia may be treated. Other malignancies with invasive metastatic potential can be treated with the subject compounds, compositions and combination therapies, including multiple myeloma. In addition, the present invention can be used to treat anemias and hematologic disorders, including anemia of chronic disease, aplastic anemia, including Fanconi's aplastic anemia; idiopathic thrombocytopenic purpura ITP); myelodysplastic syndromes (including refractory anemia, refractory anemia with ringed sideroblasts, refractory anemia with excess blasts, refractory anemia with excess blasts in transformation); myelofibrosis/myeloid metaplasia; and sickle cell vasocclusive crisis.

Various lymphoproliferative disorders also are treatable including autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma; acute lymphatic leukemias, T gamma lymphoproliferative disease, cutaneous B cell lymphoma, cutaneous T cell lymphoma (i.e., mycosis fungoides) and Sézary syndrome.

Disorders associated with transplantation are treatable with the disclosed DCL polypeptides, such as graft-versus-host disease and complications resulting from solid organ transplantation, including transplantion of heart, liver, lung, skin, kidney or other organs. DCL polypeptides may be administered, for example, to facilitate skin grafts and/or suppress differentiation of artificial skin grafts, as well as prevent or inhibit the development of bronchiolitis obliterans after lung transplantation.

The present invention also provides compositions and methods for the treatment of disorders and symptoms associated with autoimmunity and inflammation. Examples of include arthritis, diabetes, inflammatory bowel disease, systemic lupus erythmatosus, hemolytic anemia, as well as those diseases and conditions well known in the art. In one embodiment, DCL polypeptides, agonists and/or antagonists thereof are used in conjunction with one or more antigens associated with autoimmunity or inflammation whereby antigen-specific T-cell tolerance is induced to those antigens.

Administration of DCL Pharmaceutical Compositions

The present invention provide pharmaceutical compositions comprising an effective amount of a protein (DCL polypeptides, analogs, fragments, derivatives, fusion proteins, agonists and antagonists thereof) and a suitable diluent and carrier, as well as methods of using those pharmaceutical compositions for treating or preventing various diseases described above, or augmenting immune responses to those diseases. The use of DCL or homologs in conjunction with soluble cytokine receptors or cytokines, or other immunoregulatory molecules is also contemplated. Moreover, DNA encoding soluble DCL or homologs will also be useful; a tissue or organ to be transplanted can be transfected with the DNA by any method known in the art.

For therapeutic use, purified protein is administered to a patient, preferably a human, for treatment in a manner appropriate to the indication. Thus, for example, DCL protein compositions administered to regulate immune function can be given by bolus injection, continuous infusion, sustained release from implants, or other suitable technique. Typically, a therapeutic agent will be administered in the form of a composition comprising purified DCL, in conjunction with physiologically acceptable carriers, excipients or diluents. Such carriers will be nontoxic to recipients at the dosages and concentrations employed.

Ordinarily, the preparation of such protein compositions entails combining the inventive protein with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with conspecific serum albumin are exemplary appropriate diluents. Preferably, product is formulated as a lyophilizate using appropriate excipient solutions (e.g., sucrose) as diluents. Appropriate dosages can be determined in trials. The amount and frequency of administration will depend, of course, on such factors as the nature and severity of the indication being treated, the desired response, the condition of the patient, and so forth.

Suitable agonists, in addition to those described above and variants thereof, include peptides, small organic molecules, peptidomimetics, antibodies, or the like. Antibodies may be polyclonal or monoclonal; intact or truncated, e.g. F(ab′)₂, Fab, Fv; xenogeneic; allogeneic; syngeneic; or modified forms thereof, e.g. humanized, chimeric, etc.

In many cases, the agonist will be a polypeptide, an antibody or fragment thereof, etc., but other molecules that provide relatively high specificity and affinity may also be employed. Combinatorial libraries provide compounds other than oligopeptides that have the necessary binding characteristics.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

Diagnostic and Other Uses of DCL Polypeptides and Nucleic Acids

The nucleic acids encoding the DCL polypeptides provided by the present invention can be used for numerous diagnostic or other useful purposes. The nucleic acids of the invention can be used to express recombinant polypeptide for analysis, characterization or therapeutic use; as markers for tissues in which the corresponding polypeptide is preferentially expressed (either constitutively or at a particular stage of tissue differentiation or development or in disease states); as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel nucleic acids; for selecting and making oligomers for attachment to a “gene chip” or other support, including for examination of expression patterns; to raise anti-polypeptide antibodies using DNA immunization techniques; as an antigen to raise anti-DNA antibodies or elicit another immune response, and for gene therapy. Uses of DCL polypeptides and fragmented polypeptides include, but are not limited to, the following: purifying polypeptides and measuring the activity thereof; delivery agents; therapeutic and research reagents; molecular weight and isoelectric focusing markers; controls for peptide fragmentation; identification of unknown polypeptides; and preparation of antibodies. Any or all nucleic acids suitable for these uses are capable of being developed into reagent grade or kit format for commercialization as products. Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987

Probes and Primers. Among the uses of the disclosed DCL nucleic acids, and combinations of fragments thereof, is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence. The basic parameters affecting the choice of hybridization conditions and guidance for devising suitable conditions are set forth by Sambrook et al., 1989 and are described in detail above. Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified. In certain embodiments, degenerate primers can be used as probes for human or non-human genetic libraries. Such libraries would include but are not limited to cDNA libraries, genomic libraries, and even electronic EST (express sequence tag) or DNA libraries.

Diagnostics and Gene Therapy. The nucleic acids encoding DCL polypeptides, and the disclosed fragments and combinations of these nucleic acids can be used by one skilled in the art using well-known techniques to analyze abnormalities associated with the genes corresponding to these polypeptides. This enables one to distinguish conditions in which this marker is rearranged or deleted. In addition, nucleic acids of the invention or a fragment thereof can be used as a positional marker to map other genes of unknown location. The DNA can be used in developing treatments for any disorder mediated (directly or indirectly) by defective, or insufficient amounts of, the genes corresponding to the nucleic acids of the invention. Disclosure herein of native nucleotide sequences permits the detection of defective genes, and the replacement thereof with normal genes. Defective genes can be detected in in vitro diagnostic assays, and by comparison of a native nucleotide sequence disclosed herein with that of a gene derived from a person suspected of harboring a defect in this gene.

Methods of Screening for Binding Partners. The polypeptides of the present invention each can be used as reagents in methods to screen for or identify binding partners. For example, the DCL polypeptides can be attached to a solid support material and may bind to their binding partners in a manner similar to affinity chromatography. In particular embodiments, a polypeptide is attached to a solid support by conventional procedures. As one example, chromatography columns containing functional groups that will react with functional groups on amino acid side chains of polypeptides are available (Pharmacia Biotech, Inc., Piscataway, N.J.). In an alternative, a polypeptide/Fc polypeptide (as discussed above) is attached to protein A- or protein G-containing chromatography columns through interaction with the Fc moiety. The DCL polypeptides also find use in identifying cells that express a DCL binding partner. Purified DCL polypeptides are bound to a solid phase such as a column chromatography matrix or a similar suitable substrate. For example, magnetic microspheres can be coated with the polypeptides and held in an incubation vessel through a magnetic field. Suspensions of cell mixtures or cell lystes of isolated cells containing potential binding-partner-expressing cells are contacted with the solid phase having the polypeptides thereon. Cells expressing the binding partner on the cell surface bind to the fixed polypeptides, and unbound cells are washed away. In an alternative format, intracellular binding partners or substrates DCL from cell lysates bind to DCL polypeptides and unbound proteins are removed. Alternatively, DCL polypeptides can be conjugated to a detectable moiety, then incubated with cells to be tested for binding partner expression. After incubation, unbound labeled matter is removed and the presence or absence of the detectable moiety on the cells is determined. In a further alternative, mixtures of cells or cell lysates suspected of expressing the binding partner are incubated with biotinylated polypeptides. Incubation periods are typically at least one hour in duration to ensure sufficient binding. The resulting mixture then is passed through a column packed with avidin-coated beads, whereby the high affinity of biotin for avidin provides binding of the desired cells or binding partners to the beads. Procedures for using avidin-coated beads are known (see Berenson, et al. J. Cell. Biochem., 10D:239, 1986). Washing to remove unbound material, and the release of the bound cells or binding partners, are performed using conventional methods. In some instances, the above methods for screening for or identifying binding partners may also be used or modified to isolate or purify such binding partner molecules or cells expressing them.

Carriers and Delivery Agents. The polypeptides also find use as carriers for delivering agents attached thereto to cells bearing identified binding partners. The polypeptides thus can be used to deliver diagnostic or therapeutic agents to such cells (or to other cell types found to express binding partners on the cell surface) in in vitro or in vivo procedures. Detectable (diagnostic) and therapeutic agents that can be attached to a polypeptide include, but are not limited to, toxins, other cytotoxic agents, drugs, radionuclides, chromophores, enzymes that catalyze a colorimetric or fluorometric reaction, and the like, with the particular agent being chosen according to the intended application. Among the toxins are ricin, abrin, diphtheria toxin, Pseudomonas aeruginosa exotoxin A, ribosomal inactivating polypeptides, mycotoxins such as trichothecenes, and derivatives and fragments (e.g., single chains) thereof. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are ¹³¹I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu. Such agents can be attached to the polypeptide by any suitable conventional procedure. The polypeptide comprises functional groups on amino acid side chains that can be reacted with functional groups on a desired agent to form covalent bonds, for example. Alternatively, the polypeptide or agent can be derivatized to generate or attach a desired reactive functional group. The derivatization can involve attachment of one of the bifunctional coupling reagents available for attaching various molecules to polypeptides (Pierce Chemical Company, Rockford, Ill.). A number of techniques for radiolabeling polypeptides are known. Radionuclide metals can be attached to polypeptides by using a suitable bifunctional chelating agent, for example. Conjugates comprising polypeptides and a suitable diagnostic or therapeutic agent (preferably covalently linked) are thus prepared. The conjugates are administered or otherwise employed in an amount appropriate for the particular application.

Antibodies to DCL Polypeptides

Antibodies that are immunoreactive with the polypeptides of the invention are provided herein. Such antibodies specifically bind to the polypeptides via the antigen-binding sites of the antibody (as opposed to non-specific binding). In the present invention, specifically binding antibodies are those that will specifically recognize and bind with DCL polypeptides, homologues, and variants, but not with other molecules. In one preferred embodiment, the antibodies are specific for the polypeptides of the present invention and do not cross-react with other polypeptides. In this manner, the DCL polypeptides, fragments, variants, fusion polypeptides, etc., as set forth above can be employed as “immunogens” in producing antibodies immunoreactive therewith.

More specifically, the polypeptides, fragment, variants, fusion polypeptides, etc. contain antigenic determinants or epitopes that elicit the formation of antibodies. These antigenic determinants or epitopes can be either linear or conformational (discontinuous). Epitopes can be identified by any of the methods known in the art. Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kozbor et al., 1984, J. Immunol. 133:3001-3005; Cole et al., 1983, Proc. Natl. Acad. Sci. USA 80:2026-2030); and the EBV-hybridoma technique, (Cole et al., 1985, Monoclonal Antibodies And Cancer Therapy, Alan R. Liss, Inc., pp. 77-96). Hybridoma cell lines that produce monoclonal antibodies specific for the polypeptides of the invention are also contemplated herein. Such hybridomas can be produced and identified by conventional techniques. The hybridoma producing the mAb of this invention can be cultivated in vitro or in vivo. Production of high titers of mAbs in vivo makes this the presently preferred method of production. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. For the production of antibodies, various host animals can be immunized by injection with one or more of the following: a DCL polypeptide, a fragment of a DCL polypeptide, a functional equivalent of a DCL polypeptide, or a mutant form of a DCL polypeptide. Such host animals can include but are not limited to rabbits, guinea pigs, mice, and rats. Various adjuvants can be used to increase the immunologic response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies can be of any immunoglobulin class including IgG, IgM, IgE, IgA, IgD and any subclass thereof.

In addition, techniques developed for the production of “chimeric antibodies” (Takeda et al., 1985, Nature, 314: 452454; Morrison et al., 1984, Proc Natl Acad Sci USA 81: 6851-6855; Boulianne et al., 1984, Nature 312: 643-646; Neuberger et al., 1985, Nature 314: 268-270) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region. The monoclonal antibodies of the present invention also include humanized versions of murine monoclonal antibodies. Such humanized antibodies can be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Useful techniques for humanizing antibodies are also discussed in U.S. Pat. No. 6,054,297. Procedures to generate antibodies transgenically can be found in GB 2,272,440, U.S. Pat. Nos. 5,569,825 and 5,545,806, and related patents. Preferably, for use in humans, the antibodies are human or humanized; techniques for creating such human or humanized antibodies are also well known and are commercially available from, for example, Medarex Inc. (Princeton, N.J.) and Abgenix Inc. (Fremont, Calif.). In another preferred embodiment, fully human antibodies for use in humans are produced by screening a phage display library of human antibody variable domains (Vaughan et al., 1998, Nat Biotechnol. 16(6): 535-539; and U.S. Pat. No. 5,969,108).

Antigen-binding antibody fragments that recognize specific epitopes can be generated by known techniques. For example, such fragments include but are not limited to: the F(ab′)₂ fragments which can be produced by pepsin digestion of the antibody molecule and the Fab fragments which can be generated by reducing the disulfide bridges of the (ab′)₂ fragments. Alternatively, Fab expression libraries can be constructed (Huse et al., 1989, Science, 246:1275-1281) to allow rapid and easy identification of monoclonal Fab fragments with the desired specificity. Techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can also be adapted to produce single chain antibodies against DCL gene products. Single chain antibodies are formed by linking the heavy and light chain fragments of the Fv region via an amino acid bridge, resulting in a single chain polypeptide. Such single chain antibodies can also be useful intracellularly (i.e., as intrabodies), for example as described by Marasco et al. (J. Immunol. Methods 231:223-238, 1999) for genetic therapy in HIV infection. In addition, antibodies to the DCL polypeptide can, in turn, be utilized to generate anti-idiotype antibodies that “mimic” the DCL polypeptide and that may bind to the DCL polypeptide's binding partners using techniques well known to those skilled in the art. (See, e.g., Greenspan & Bona, 1993, FASEB J 7(5):437444; and Nissinoff, 1991, J. Immunol. 147(8):2429-2438).

Antibodies that are immunoreactive with the polypeptides of the invention include bispecific antibodies (i.e., antibodies that are immunoreactive with the polypeptides of the invention via a first antigen binding domain, and also immunoreactive with a different polypeptide via a second antigen binding domain). A variety of bispecific antibodies have been prepared, and found useful both in vitro and in vivo (see, for example, U.S. Pat. No. 5,807,706; and Cao and Suresh, 1998, Bioconjugate Chem 9: 635-644). Numerous methods of preparing bispecific antibodies are known in the art, including the use of hybrid-hybridomas such as quadromas, which are formed by fusing two differed hybridomas, and triomas, which are formed by fusing a hybridoma with a lymphocyte (Milstein and Cuello, 1983, Nature 305: 537-540; U.S. Pat. No. 4,474,893; and U.S. Pat. No. 6,106,833). U.S. Pat. No. 6,060,285 discloses a process for the production of bispecific antibodies in which at least the genes for the light chain and the variable portion of the heavy chain of an antibody having a first specificity are transfected into a hybridoma cell secreting an antibody having a second specificity. Chemical coupling of antibody fragments has also been used to prepare antigen-binding molecules having specificity for two different antigens (Brennan et al., 1985, Science 229: 81-83; Glennie et al., J. Immunol., 1987, 139:2367-2375; and U.S. Pat. No. 6,010,902). Bispecific antibodies can also be produced via recombinant means, for example, by using the leucine zipper moieties from the Fos and Jun proteins (which preferentially form heterodimers) as described by Kostelny et al. (J. Immunol. 148:1547-4553; 1992). U.S. Pat. No. 5,582,996 discloses the use of complementary interactive domains (such as leucine zipper moieties or other lock and key interactive domain structures) to facilitate heterodimer formation in the production of bispecific antibodies. Tetravalent, bispecific molecules can be prepared by fusion of DNA encoding the heavy chain of an F(ab′)₂ fragment of an antibody with either DNA encoding the heavy chain of a second F(ab′)₂ molecule (in which the CH1 domain is replaced by a CH3 domain), or with DNA encoding a single chain FV fragment of an antibody, as described in U.S. Pat. No. 5,959,083. Expression of the resultant fusion genes in mammalian cells, together with the genes for the corresponding light chains, yields tetravalent bispecific molecules having specificity for selected antigens. Bispecific antibodies can also be produced as described in U.S. Pat. No. 5,807,706. Generally, the method involves introducing a protuberance (constructed by replacing small amino acid side chains with larger side chains) at the interface of a first polypeptide and a corresponding cavity (prepared by replacing large amino acid side chains with smaller ones) in the interface of a second polypeptide. Moreover, single-chain variable fragments (sFvs) have been prepared by covalently joining two variable domains; the resulting antibody fragments can form dimers or trimers, depending on the length of a flexible linker between the two variable domains (Kortt et al., 1997, Protein Engineering 10:423-433).

Screening procedures by which such antibodies can be identified are well known, and can involve immunoaffinity chromatography, for example. Antibodies can be screened for agonistic (i.e., ligand-mimicking) properties. Such antibodies, upon binding to cell surface DCL, induce biological effects (e.g., transduction of biological signals) similar to the biological effects induced when the DCL binding partner binds to cell surface DCL. Agonistic antibodies can be used to induce DCL-mediated cell stimulatory pathways or intercellular communication. Bispecific antibodies can be identified by screening with two separate assays, or with an assay wherein the bispecific antibody serves as a bridge between the first antigen and the second antigen (the latter is coupled to a detectable moiety).

Those antibodies that can block binding of the DCL polypeptides of the invention to binding partners for DCL can be used to inhibit DCL-mediated intercellular communication or cell stimulation that results from such binding. Such blocking antibodies can be identified using any suitable assay procedure, such as by testing antibodies for the ability to inhibit binding of DCL to certain cells expressing an DCL binding partner. Alternatively, blocking antibodies can be identified in assays for the ability to inhibit a biological effect that results from binding of soluble DCL to target cells. Antibodies can be assayed for the ability to inhibit DCL binding partner-mediated cell stimulatory pathways, for example. Such an antibody can be employed in an in vitro procedure, or administered in vivo to inhibit a biological activity mediated by the entity that generated the antibody. Disorders caused or exacerbated (directly or indirectly) by the interaction of DCL with cell surface binding partner receptor thus can be treated. A therapeutic method involves in vivo administration of a blocking antibody to a mammal in an amount effective in inhibiting DCL binding partner-mediated biological activity. Monoclonal antibodies are generally preferred for use in such therapeutic methods. In one embodiment, an antigen-binding antibody fragment is employed. Compositions comprising an antibody that is directed against DCL and a physiologically acceptable diluent, excipient, or carrier, are provided herein. Suitable components of such compositions are as described below for compositions containing DCL polypeptides.

Also provided herein are conjugates comprising a detectable (e.g., diagnostic) or therapeutic agent, attached to the antibody. Examples of such agents are presented above. The conjugates find use in in vitro or in vivo procedures. The antibodies of the invention can also be used in assays to detect the presence of the polypeptides or fragments of the invention, either in vitro or in vivo. The antibodies also can be employed in purifying polypeptides or fragments of the invention by immunoaffinity chromatography.

### EXAMPLES

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade, and pressure is at or near atmospheric.

### EXAMPLE 1

### Generation of Bone-Marrow Derived Murine DCS and Preparation of Labeled Targets for Affymetrix Gene Chip™ Microarray Experiments

Mice

Female C57BU10 mice (8 to 12 weeks of age) were obtained from Taconic (Germantown, N.Y.). All mice were housed under specific pathogen-free conditions.

Cell Preparations:

Bone marrow (BM) cells were isolated by flushing femurs with 2 ml phosphate-buffered saline (PBS) supplemented with 2% heat-inactivated fetal bovine serum (FBS) (Gibco BRL Life Technologies, Gaithersburg, Md.). The BM cells were centrifuged once and then resuspended in tris-ammonium chloride at 37° C. for 2 minutes to lyse red blood cells. The cells were centrifuged again and then resuspended in culture medium (CM) consisting of McCoy's medium supplemented with essential and nonessential amino acids, 1 mmol/l sodium pyruvate, 2.5 mmol/l Hepes buffer pH 7.4, vitamins, 5.5×10⁻⁵ mol/l 2-mercaptoethanol (2-ME), 100 U/ml penicillin, 100 μg/ml streptomycin, 0.3 mg/ml L-glutamine (PSG), and 10% FBS (all media reagents from Gibco).

DC Cultures:

BM cells were cultured in CM containing 200 ng/ml (180 units/ml) human Flt-3L (Amgen Corp.) for 9 days at 1×10⁶/ml, in 6-well plates (Costar/Corning Incorp., Acton, Mass.). Cultures were incubated at 37° C. in a humidified atmosphere containing 5% CO₂ in air. DCs were harvested from the cultures by vigorously pipetting and removing nonadherent cells, then washing each well 2 times with room temperature PBS without Ca⁺⁺ or Mg⁺⁺ to remove loosely adherent cells, which were pooled with the nonadherent fraction.

It is well known in the art that the overall number of functionally mature dendritic cells in the host may be expanded through the prior administration of a suitable growth factor, which growth factor may be one or more of Flt3-L; GM-CSF; G-CSF; GM-CSF+IL-4; GM-CSF+IL-3; etc. For example, Flt3-L (Amgen Corp., Seattle, Wash.) has been found to stimulate the generation of large numbers of functionally mature dendritic cells, both in vivo and in vitro (U.S. Ser. No. 08/539,142, filed Oct. 4, 1995). Flt3-L refers to a genus of polypeptides that are described in EP 0627487 A2 and in WO 94/28391, both incorporated herein by reference. Other useful cytokines include granulocyte-macrophage colony stimulating factor (GM-CSF; described in U.S. Pat. Nos. 5,108,910, and 5,229,496 each of which is incorporated herein by reference). Moreover, GM-CSP/IL-3 fusion proteins (i.e., a C-terminal to N-terminal fusion of GM-CSF and IL-3) may be used. Such fusion proteins are well known in the art and are described in U.S. Pat. Nos. 5,199,942; 5,108,910 and 5,073,627, each of which is incorporated herein by reference. Various routes and regimens for delivery may be used, as known and practiced in the art. For example, where the agent is Flt3-L, the Flt3-L may be administered daily, where the dose is from about 1 to 100 mg/kg body weight, more usually from about 10 to about 50 mg/kg body weight. Administration may be at a localized site, e.g. subcutaneous, or systemic, e.g. intraperitoneal, intravenous, etc.

The Flt3-L-derived DCs were cultured for 10 days. On day ten, the cultures were stimulated for 4 hours with the following stimuli/conditions: (a) 10 ng/ml recombinant murine GM-CSF, 1000 U/ml human; (b) 500 U/ml IFN-A/D (Genzyme, Cambridge, Mass.); (c) 1 μg/ml Escherichia coli (E coli)(0217:B8)-derived LPS (Difco, Detroit, Mich.) and (d) no stimulus.

RNA Isolation:

After the 4 hr stimulation, the cells were immediately lyzed in Trizol™ (Gibco BRL Life Technologies) and the RNA was isolated according to manufacturer's recommendations. The isolated RNA was further isolated using the RNeasy™ kit from Qiagen (Qiagen Inc., Valencia, Calif.).

Preparation of Labeled RNA Targets for Hybridization to Affymetrix™ Arrays:

The preparation of the target RNAs and hybridization to the microarray chips was performed essentially as described in the Affymetrix protocols (Affymetrix Corp., Santa Clara, Calif.), which are incorporated herein by reference. Briefly, the target sample was prepared using 10 ug of total RNA, which was first converted to single-stranded cDNA using Superscript II™ reverse transcriptase (Gibco BRL Life Technologies) and a primer encoding the bacteriophage T7 RNA polymerase promoter. The single-stranded cDNA was then converted to double-stranded cDNA. The T7 promoter was used to generate a labeled cRNA target in a reaction containing 17 RNA polymerase and biotinylated nucleotide triphosphates. After purification, the cRNA was chemically fragmented to an average length of 50-200 bases and hybridized overnight at 45° C. to Affymetrix gene chips. This cRNA is complementary to short DNA probes synthesized on the Affymetrix Gene Chip™ arrays. After hybridization, the chips were processed in the Affymetrix fluidics station. They were washed, stained with streptavidin phycoerythrin (SAPE), followed by biotinylated goat anti-streptavidin, and a second round of SAPE.

### EXAMPLE 2

### Preparation of Antibodies

The following example illustrates a method for preparing monoclonal antibodies that bind DCL polypeptides. Other conventional techniques may be used, such as those described in U.S. Pat. No. 4,411,993. Immunogen preparation, choice of adjuvant and immunization protocol are methods that are well known in the art and may be found, for example in Antibodies: A Laboratory Manual, Harlow and Land (eds.), Cold Spring Harbor Laboratory Press, Cold Spring Harbor, N.Y., (1988). Suitable immunogens that may be employed in generating such antibodies include, but are not limited to, purified DCL polypeptides, an immunogenic fragment thereof, and cells expressing high levels of DCL polypeptides or an immunogenic fragment thereof. DNA encoding one or more DCL polypeptides may also be used as an immunogen, for example, as reviewed by Pardoll and Beckerleg in Immunity 3: 165, 1995.

Rodents (BALB/c mice or Lewis rats, for example) are immunized with a DCL polypeptide immunogen emulsified in an adjuvant (such as complete or incomplete Freund's adjuvant, alum, or another adjuvant, such as Ribi adjuvant R700 (Ribi, Hamilton, Mont.)), and injected in amounts ranging from 10-100 micrograms subcutaneously or intraperitoneally. DNA may be given intradermally (Raz et al., 1994, Proc. Natl. Acad. Sci. USA 91: 9519) or intamuscularly (Wang et al., 1993, Proc. Natl. Acad. Sci. USA 90: 4156); saline has been found to be a suitable diluent for DNA-based antigens. Ten days to three weeks days later, the immunized animals are boosted with additional immunogen and periodically boosted thereafter on a weekly, biweekly or every third week immunization schedule.

Serum samples are periodically taken by retro-orbital bleeding or tail-tip excision to test for DCL polypeptide antibodies by dot-blot assay, ELISA (enzyme-linked immunosorbent assay), immunoprecipitation, or other suitable assays, such as FACS analysis of inhibition of binding of DCL polypeptide to a DCL polypeptide binding partner. Following detection of an appropriate antibody titer, positive animals are provided one last intravenous injection of DCL polypeptide in saline. Three to four days later, the animals are sacrificed, and spleen cells are harvested and fused to a murine myeloma cell line, e.g., NS1 or preferably P3X63Ag8.653 (ATCC CRL-1580). These cell fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells may be screened by ELISA for reactivity against purified DCL polypeptide by adaptations of the techniques disclosed in Engvall et al., (Immunochem. 8: 871, 1971) and in U.S. Pat. No. 4,703,004. A preferred screening technique is the antibody capture technique described in Beckmann et al., (J. Immunol. 144: 4212, 1990). Positive hybridoma cells can be injected intraperitoneally into syngeneic rodents to produce ascites containing high concentrations (for example, greater than 1 milligram per milliliter) of anti-DCL polypeptide monoclonal antibodies. Alternatively, hybridoma cells can be grown in vitro in flasks or roller bottles by various techniques. Monoclonal antibodies can be purified by ammonium sulfate precipitation, followed by gel exclusion chromatography. Alternatively, affinity chromatography based upon binding of antibody to protein A or protein G can also be used, as can affinity chromatography based upon binding to DCL polypeptide.

### EXAMPLE 3

### Antisense Inhibition of DCL Nucleic Acid Expression

In accordance with the present invention, a series of oligonucleotides are designed to target different regions of the DCL mRNA molecule, using the nucleotide sequence of SEQ ID NO:1, 5, 9, 11, 15, 17, 21 and 23 as the basis for the design of the oligonucleotides. The oligonucleotides are selected to be approximately 10, 12, 15, 18, or more preferably 20 nucleotide residues in length, and to have a predicted hybridization temperature that is at least 37 degrees C. Preferably, the oligonucleotides are selected so that some will hybridize toward the 5′ region of the mRNA molecule, others will hybridize to the coding region, and still others will hybridize to the 3′ region of the mRNA molecule. Methods such as those of Gray and Clark (U.S. Pat. Nos. 5,856,103 and 6,183,966) can be used to select oligonucleotides that form the most stable hybrid structures with target sequences, as such oligonucleotides are desirable for use as antisense inhibitors.

The oligonucleotides may be oligodeoxynucleotides, with phosphorothioate backbones (internucleoside linkages) throughout, or may have a variety of different types of internucleoside linkages. Generally, methods for the preparation, purification, and use of a variety of chemically modified oligonucleotides are described in U.S. Pat. No. 5,948,680. As specific examples, the following types of nucleoside phosphoramidites may be used in oligonucleotide synthesis: deoxy and 2′-alkoxy amidites; 2′-fluoro amidites such as 2′-fluorodeoxyadenosine amidites, 2′-fluorodeoxyguanosine, 2′-fluorouridine, and 2′-fluorodeoxycytidine; 2′-O-(2-methoxyethyl)-modified amidites such as 2,2′-anhydro[1-(beta-D-arabino-furanosyl)-5-methyluridine], 2′-O-methoxyethyl-5-methyluridine, 2′-O-methoxyethyl-5′-O-dimethoxytrityl-5-methyluridine, 3′-O-acetyl-2′-O-methoxy-ethyl-5′-O-dimethoxytrityl-5-methyluridine, 3′-O-acetyl-2′-O-methoxyethyl-5′-O-dimethoxytrityl-5-methyl-4-triazoleuridine, 2′-O-methoxyethyl-5′-O-dimethoxytrityl-5-methylcytidine, N4-benzoyl-2′-O-methoxyethyl-5′-O-dimethoxytrityl-5-methylcytidine, and N4-benzoyl-2′-O-methoxyethyl-5′-O-di-methoxytrityl-5-methylcytidine-3′-amidite; 2′-O-(aminooxyethyl) nucleoside amidites and 2′-O-(dimethylaminooxyethyl) nucleoside amidites such as 2′-(dimethylaminooxyethoxy) nucleoside amidites, 5′-O-tert-butyldiphenylsilyl-O²-2′-anhydro-5-methyluridine, 5′-O-tert-butyl-diphenylsilyl-2′-O-(2-hydroxyethyl)-5-methyluridine, 2′-O-([2-phthalimidoxy)ethyl]-5′-t-butyldiphenyl-silyl-5-methyluridine, 5′-O-tert-butyldiphenylsilyl-2′-O-[(2-formadoximinooxy)ethyl]-5-methyluridine, 5′-O-tert-butyldiphenylsilyl-2′-O-[N,N-dimethylaminooxyethyl]-5-methyluridine, 2′-β-(dimethylaminooxy-ethyl)-5-methyluridine, 5′-O-DMT-2′-O-(dimethylaminooxyethyl)-5-methyluridine, and 5′-O-DMT-2′-O-(2-N,N-dimethylaminooxyethyl)-5-methyluridine-3′-[(2-cyanoethyl)-N,N-diisopropylphosphor-amidite]; and 2′-(aminooxyethoxy) nucleoside amidites such as N2-isobutyryl-6-O-diphenyl-carbamoyl-2′-O-(2-ethylacetyl)-5′-O-(4,4′-dimethoxytrityl)guanosine-3′-[(2-cyanoethyl)-N,N-diiso-propylphosphoramidite].

Modified oligonucleosides may also be used in oligonucleotide synthesis, for example methylenemethylimino-linked oligonucleosides, also called MMI-linked oligonucleosides; methylene-dimethylhydrazo-linked oligonucleosides, also called MDH-linked oligonucleosides; methylene-carbonylamino-linked oligonucleosides, also called amide-3-linked oligonucleosides; and methylene-aminocarbonyl-linked oligonucleosides, also called amide-4-linked oligonucleosides, as well as mixed backbone compounds having, for instance, alternating MMI and P═O or P═S linkages, which are prepared as described in U.S. Pat. Nos. 5,378,825, 5,386,023, 5,489,677, 5,602,240 and 5,610,289. Formacetal- and thioformacetal-linked oligonucleosides may also be used and are prepared as described in U.S. Pat. Nos. 5,264,562 and 5,264,564; and ethylene oxide linked oligonucleosides may also be used and are prepared as described in U.S. Pat. No. 5,223,618. Peptide nucleic acids (PNAs) may be used as in the same manner as the oligonucleotides described above, and are prepared in accordance with any of the various procedures referred to in Peptide Nucleic Acids (PNA): Synthesis, Properties and Potential Applications, Bioorganic & Medicinal Chemistry, 1996, 4, 5-23; and U.S. Pat. Nos. 5,539,082, 5,700,922, and 5,719,262.

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Some examples of different types of chimeric oligonucleotides are: [2′-O-Me]-[2′-deoxy]-[2′-O-Me] chimeric phosphorothioate oligonucleotides, [2′-O-(2-methoxyethyl)]-[2′-deoxy]-[2′-O-(methoxyethyl)] chimeric phosphorothioate oligonucleotides, and [2′-O-(2-methoxy-ethyl)phosphodiester]-[2′-deoxy phosphoro-thioate]-[2′-O-(2-methoxyethyl)phosphodiester] chimeric oligonucleotides, all of which may be prepared according to U.S. Pat. No. 5,948,680. In one preferred embodiment, chimeric oligonucleotides (“gapmers”) 18 nucleotides in length are utilized, composed of a central “gap” region consisting of ten 2′-deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. Other chimeric oligonucleotides, chimeric oligonucleosides, and mixed chimeric oligonucleotides/oligonucleosides are synthesized according to U.S. Pat. No. 5,623,065.

Oligonucleotides are preferably synthesized via solid phase P(III) phosphoramidite chemistry on an automated synthesizer. The concentration of oligonucleotide in each well is assessed by dilution of samples and UV absorption spectroscopy. The full-length integrity of the individual products is evaluated by capillary electrophoresis, and base and backbone composition is confirmed by mass analysis of the compounds utilizing electrospray-mass spectroscopy.

The effect of antisense compounds on target nucleic acid expression can be tested in any of a variety of cell types provided that the target nucleic acid is present at measurable levels. This can be routinely determined using, for example, PCR or Northern blot analysis. Cells are routinely maintained for up to 10 passages as recommended by the supplier. When cells reached 80% to 90% confluency, they are treated with oligonucleotide. For cells grown in 96-well plates, wells are washed once with 200 microliters OPTI-MEM-1 reduced-serum medium (Gibco BRL) and then treated with 130 microliters of OPTI-MEM-1 containing 3.75 g/mL LIPOFECTIN (Gibco BRL) and the desired oligonucleotide at a final concentration of 150 nM. After 4 hours of treatment, the medium is replaced with fresh medium. Cells are harvested 16 hours after oligonucleotide treatment. Preferably, the effect of several different oligonucleotides should be tested simultaneously, where the oligonucleotides hybridize to different portions of the target nucleic acid molecules, in order to identify the oligonucleotides producing the greatest degree of inhibition of expression of the target nucleic acid.

Antisense modulation of DCL nucleic acid expression can be assayed in a variety of ways known in the art. For example, DCL mRNA levels can be quantitated by, e.g., Northern blot analysis, competitive polymerase chain reaction (PCR), or real-time PCR. Real-time quantitative PCR is presently preferred. RNA analysis can be performed on total cellular RNA or poly(A)+mRNA. Methods of RNA isolation and Northern blot analysis are taught in, for example, Ausubel, F. M. et al., Current Protocols in Molecular Biology, Volume 1, pp. 4.1.14.2.9 and 4.5.14.5.3, John Wiley & Sons, Inc., 1996. Real-time quantitative (PCR) can be conveniently accomplished using the commercially available ABI PRISM 7700 Sequence Detection System, available from PE-Applied Biosystems, Foster City, Calif. and used according to manufacturer's instructions. This fluorescence detection system allows high-throughput quantitation of PCR products. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or PAM, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples. Other methods of quantitative PCR analysis are also known in the art. DCL protein levels can be quantitated in a variety of ways well known in the art, such as immunoprecipitation, Western blot analysis (immunoblotting), ELISA, or fluorescence-activated cell sorting (FACS). Antibodies directed to DCL polypeptides can be prepared via conventional antibody generation methods such as those described herein. Immunoprecipitation methods, Western blot (immunoblot) analysis, and enzyme-linked immunosorbent assays (ELISA) are standard in the art (see, for example, Ausubel, F. M. et al., Current Protocols in Molecular Biology, Volume 2, pp. 10.16.1-10.16.11, 10.8.1-10.8.21, and 11.2.1-11.2.22, John Wiley & Sons, Inc., 1991).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### SEQUENCE IDENTIFIERS

SEQ ID NO:1 is the full-length cDNA sequence for DCL 1.

SEQ ID NO:2 is the full-length ORF amino acid sequence for DCL 1.

SEQ ID NO:3 is the sense-oriented PCR primer for cloning DCL 1.

SEQ ID NO:4 is the antisense-oriented PCR primer for cloning DCL 1.

SEQ ID NO:5 is the full-length cDNA sequence for DCL 2.

SEQ ID NO:6 is the full-length ORF amino acid sequence for DCL 2.

SEQ ID NO:7 is the sense-oriented PCR primer for cloning DCL 2.

SEQ ID NO:8 is the antisense-oriented PCR primer for cloning DCL 2.

SEQ ID NO:9 is the cDNA sequence for an alternative splice variant of DCL 2 (exon 3 deleted).

SEQ ID NO:10 is the amino acid sequence for an alternative splice variant of DCL 2 (exon 3 deleted).

SEQ ID NO:11 is the full-length cDNA sequence for DCL 3.

SEQ ID NO:12 is the full-length ORF amino acid sequence for DCL 3.

SEQ ID NO:13 is the sense-oriented PCR primer for cloning DCL 3.

SEQ ID NO:14 is the antisense-oriented PCR primer for cloning DCL 3.

SEQ ID NO:15 is the cDNA sequence for an alternative splice variant of DCL 3 (exons 4 and 5 deleted).

SEQ ID NO:16 is the amino acid sequence for an alternative splice variant of DCL 3 (exons 4 and 5 deleted).

SEQ ID NO:17 is the full-length cDNA sequence for DCL 4.

SEQ ID NO:18 is the full-length ORF amino acid sequence for DCL 4.

SEQ ID NO:19 is the sense-oriented PCR primer for cloning DCL 4.

SEQ ID NO:20 is the antisense-oriented PCR primer for cloning DCL 4.

SEQ ID NO:21 is the cDNA sequence for an alternative splice variant of DCL 4 (exon 4 deleted).

SEQ ID NO:22 is the amino acid sequence for an alternative splice variant of DCL 4 (exon 4 deleted).

SEQ ID NO:23 is the full-length cDNA sequence for DCL 5.

SEQ ID NO:24 is the full-length ORF amino acid sequence for DCL 5.

SEQ ID NO:25 is the sense-oriented PCR primer for cloning DCL 5.

SEQ ID NO:26 is the antisense-oriented PCR primer for cloning DCL 5.

What is claimed is:

1. An isolated polypeptide comprising an amino acid sequence selected from the group consisting of:
(a) the amino acid sequence of SEQ ID NO:2;
(b) an amino acid sequence selected from the group consisting of: amino acids 1 through 245 of SEQ ID NO:2 and amino acids 77 through 245 of SEQ ID NO:2;
(c) the amino acid sequence of SEQ ID NO:2 comprising all or part of the extracellular domain having at least one DCL activity;
(d) fragments of the amino acid sequences of any of (a)-(c) comprising at least 25 contiguous amino acids having at least one DCL activity;
(e) fragments of the amino acid sequences of any of (a)-(c) comprising at least 25 contiguous amino acids having a C-type lectin domain;
(f) fragments of the amino acid sequences of any of (a)-(c) comprising at least 25 contiguous amino acids having an immunoreceptor tyrosine-based inhibitory-like motif (ITIM) amino acid sequences;
(g) fragments of the amino acid sequences of any of (a)-(c) comprising at least 25 contiguous amino acids that are immunogenic;
(h) amino acid sequences comprising at least 25 amino acids and sharing amino acid identity with the amino acid sequences of any of (a)-(g), wherein the percent amino acid identity is at least 80%, wherein the amino acid sequences have at least one DCL activity; and
(i) amino acid sequences of any of (a)-(c) comprising at least 20 amino acids having at least one modification selected from the group consisting of amino acid substitutions, amino acid insertions, amino acid deletions, C-terminal truncation, and N-terminal truncation, wherein the amino acid sequences have at least one DCL activity.

2. An isolated polynucleotide encoding a polypeptide of claim 1.

3. The polynucleotide of claim 2 comprising a nucleotide sequence selected from the group consisting of:
(a) SEQ ID NO:1;
(b) nucleotides 1 through 738 of SEQ ID NO:1; and
(c) allelic variants of (a)-(b).

4. An isolated genomic polynucleotide corresponding to the polynucleotide of any of the claims 2 and 3.

5. An isolated polynucleotide, having a length of at least 15 nucleotides, that hybridizes under conditions of moderate stringency to a complementary nucleic acid of the polynucleotide of claim 3, wherein the polynucleotide encodes a polypeptide having at least one DCL activity.

6. An isolated polynucleotide comprising a nucleotide sequence that shares nucleotide sequence identity with the nucleotide sequences of the nucleic acids of claim 3, wherein the percent nucleotide sequence identity is at least 80%, wherein the polynucleotide encodes a polypeptide having at least one DCL activity.

7. An expression vector comprising at least one polynucleotide according to any of claims 2 through 6.

8. A transformed host cell comprising at least one polynucleotide according to any of claims 2 through 6.

9. The transformed host cell of claim 8, wherein the transformed host cell is selected from the group consisting of prokaryotic cells, eukaryotic cells, bacterial cells, yeast cells, insect cells, and mammalian cells such as human, monkey, ape and rodent.

10. A process for producing a polypeptide encoded by the polynucleotide of any of claims 2 through 6, comprising culturing a transformed host cell under conditions promoting expression of said polypeptide, wherein the transformed host cell comprises at least one polynucleotide according to any of claims 2 through 6.

11. The process of claim 10 further comprising purifying said polypeptide.

12. The polypeptide produced by the process of claim 11.

13. An isolated antibody that binds to the polypeptide of claim 1.

14. The antibody of claim 13 wherein the antibody is a monoclonal antibody.

15. The antibody of claim 14 wherein the monoclonal antibody is a human or humanized monoclonal antibody.

16. The antibody of claim 15 wherein the antibody agonizes one or more DCL activities of the polypeptide of claim 1.

17. An isolated polypeptide comprising an amino acid sequence selected from the group consisting of:
(a) the amino acid sequence of SEQ ID NO:5;
(b) an amino acid sequence selected from the group consisting of: amino acids 1 through 215 of SEQ ID NO:5 and amino acids 42 through 215 of SEQ ID NO:5;
(c) the amino acid sequence of SEQ ID NO:5 comprising all or part of the extracellular domain having at least one DCL activity;
(d) fragments of the amino acid sequences of any of (a)-(c) comprising at least 25 contiguous amino acids having at least one DCL activity;
(e) fragments of the amino acid sequences of any of (a)-(c) comprising at least 25 contiguous amino acids having a C-type lectin domain;
(f) fragments of the amino acid sequences of any of (a)-(c) comprising at least 25 contiguous amino acids that are immunogenic;
(g) amino acid sequences comprising at least 25 amino acids and sharing amino acid identity with the amino acid sequences of any of (a)-(f), wherein the percent amino acid identity is at least 80%, wherein the amino acid sequences have at least one DCL activity; and
(h) amino acid sequences of any of (a)-(c) comprising at least 20 amino acids having at least one modification selected from the group consisting of amino acid substitutions, amino acid insertions, amino acid deletions, C-terminal truncation, and N-terminal truncation, wherein the amino acid sequences have at least one DCL activity.

18. An isolated polynucleotide encoding a polypeptide of claim 17.

19. The polynucleotide of claim 18 comprising a nucleotide sequence selected from the group consisting of:
(a) SEQ ID NO:23;
(b) nucleotides 1 through 648 of SEQ ID NO:23; and
(c) allelic variants of (a)-(b).

20. An isolated genomic polynucleotide corresponding to the polynucleotide of any of claims 18 and 19.

21. An isolated polynucleotide, having a length of at least 15 nucleotides, that hybridizes under conditions of moderate stringency to a complementary nucleic acid of the polynucleotide of claim 19, wherein the polynucleotide encodes a polypeptide having at least one DCL activity.

22. An isolated polynucleotide comprising a nucleotide sequence that shares nucleotide sequence identity with the nucleotide sequences of the nucleic acids of claim 19, wherein the percent nucleotide sequence identity is at least 80%, wherein the polynucleotide encodes a polypeptide having at least one DCL activity.

23. An expression vector comprising at least one polynucleotide according to any of claims 18 through 22.

24. A transformed host cell comprising at least one polynucleotide according to any of claims 18 through 22.

25. The transformed host cell of claim 24, wherein the transformed host cell is selected from the group consisting of prokaryotic cells, eukaryotic cells, bacterial cells, yeast cells, insect cells, and mammalian cells such as human, monkey, ape and rodent.

26. A process for producing a polypeptide encoded by the polynucleotide of any of claims 18 through 22, comprising culturing a transformed host cell under conditions promoting expression of said polypeptide, wherein the transformed host cell comprises at least one polynucleotide according to any of claims 18 through 22.

27. The process of claim 26 further comprising purifying said polypeptide.

28. The polypeptide produced by the process of claim 27.

29. An isolated antibody that binds to the polypeptide of claim 17.

30. The antibody of claim 29 wherein the antibody is a monoclonal antibody.

31. The antibody of claim 30 wherein the monoclonal antibody is a human or humanized monoclonal antibody.

32. The antibody of claim 31 wherein the antibody agonizes one or more DCL activities of the polypeptide of claim 17.

33. The polypeptide of claims 1 or 17, wherein the polypeptide has an activity selected from the group consisting of antigen binding, antigen internalization, antigen processing and antigen presentation; antigen presenting cell (APC) activation, APC differentiation, APC maturation, APC homing and APC transmigration; cell to cell interactions including binding and modulation of intracellular signaling pathways in either an excitatory or inhibitory manner; extracellular communication through secretion of soluble factors; C-type lectin activity; carbohydrate recognition domain activity;
aspartyl protease activity and immunoreceptor tyrosine-based inhibitory-like motif (ITIM) activity.

34. A method for identifying compounds that modulate DCL polypeptide activity comprising
(a) mixing a test compound with the polypeptide of claim 1 or claim 17; and
(b) determining whether the test compound alters the DCL polypeptide activity of said polypeptide.

35. A method for identifying compounds that inhibit the binding activity of DCL polypeptides comprising
(a) mixing a test compound with the polypeptide of claim 1 or claim 17 and a binding partner of said polypeptide; and
(b) determining whether the test compound inhibits the binding activity of said polypeptide.

36. A method for increasing DCL activity comprising providing at least one compound selected from the group consisting of the polypeptide of any of claims 1 and 17 and agonists of said polypeptides.

37. The method of claim 36, wherein the agonists is selected from the group consisting of an antibody, a peptide, peptidomimetic, mimotope or a peptibody.

38. A method for decreasing one or more DCL activities comprising providing at least one antagonist of the polypeptide of any of claims 1 and 17.

39. The method of claim 38, wherein the antagonists is selected from the group consisting of an antibody, a peptide, peptidomimetic, mimotope or a peptibody.

40. A method for treating an infectious disease comprising administering one or more polypeptides according to any of claims 1 and 17 coupled to one or more antigens from infectious agents.

41. A method of augmenting an immune response to an infectious agent comprising administering one or more polypeptides according to any of claims 1 and 17 coupled to one or more antigens from infectious agents.

42. A method for treating cancer comprising administering one or more polypeptides according to any of claims 1 and 17 coupled to one or more tumor antigens.

43. A method of augmenting an immune response to cancer comprising administering one or more polypeptides according to any of claims 1 and 17 coupled to one or more tumor antigens.

44. A method of inducing antigen-specific tolerance in cells of the immune system comprising administering one or more polypeptides according to any of claims 1 and 17 coupled to one or more antigens associated with autoimmunity or inflammation.


SEQUENCE LISTING (26 sequences)

SEQ ID NO: 1 - DNA, 738 bp, Mus sp.
  CDS (1)..(738)
atggcattaccaaacatttatactgacgtgaacttcaaaaatcaacctgtttcctcaggcctcatctcagactcgtcttcatgtaccgtctcagactcgtcttcagctctcccaaagaagaccactattcacaaaagtaaccctggctttcccaggctgcttcttgccttgtggatatttttcctgctgttggcaatcttattctctgttgctctgatcattttatttcaaatgtattctgatctccttgaagaaaaatatactctagaacgactgaatcacgcaagattgcattgtgtaaaaaaccactcgtctgtagaagacaaagtctggagctgttgtccaaagaattggaagccatttgattcccactgctacttcacttcccgtgacactgcatcctggagtaagagtgaagagaagtgctccctcaggggtgctcatctgctggtgatccagagccaggaagagcaggatttcatcaccaacactctgaaccctcgtgctgcttattatgtggggctgtcagatccaaagggccatggacaatggcagtgggttgatcagacaccatatgatcaaaatgccacatcctggcactcagatgaacccagtggcaacactgaattttgtgttgtgctaagttatcatccaaacgttaaaggatggggctggagtgtcgccccttgtgatggtgatcataggttgatttgtgagatgaggcagctctatgtatga

SEQ ID NO: 2 - protein, 245 aa, Mus sp.
MALPNIYTDVNFKNQPVSSGLISDSSSCTVSDSSSALPKKTTIHKSNPGFPRLLLALWIFFLLLAILFSVALIILFQMYSDLLEEKYTLERLNHARLHCVKNHSSVEDKVWSCCPKNWKPFDSHCYFTSRDTASWSKSEEKCSLRGAHLLVIQSQEEQDFITNTLNPRAAYYVGLSDPKGHGQWQWVDQTPYDQNATSWHSDEPSGNTEFCVVLSYHPNVKGWGWSVAPCDGDHRLICEMRQLYV

SEQ ID NO: 3 - DNA, 33 bp, Artificial Sequence
  Oligonucleotide
atggcattaccaaacatttatactgacgtgaac

SEQ ID NO: 4 - DNA, 31 bp, Artificial Sequence
  Oligonucleotide
atgcttcgttcatacatagagctgcctcatc

SEQ ID NO: 5 - DNA, 714 bp, Mus sp.
  CDS (1)..(714)
atgttttcagaaaacatttatgttaacacgaacttcaaaaataaagttgactcctcagacatcgacacagactcttggccagctccccaaaggaagaacacgtctcagaaaagttgtcacaaattctctaaggtcctctttacctcactcataatctatttcctgctgttgacaatcttattctccggtgctctgatcactttgtttacaaaatattctcagcttcttgaagaaaaaatgattataaaagaactgaactatactgaattggagtgtacaaaatgggcttcactcttggaagacaaagtctggagctgttgcccaaaggattggaagccgtttggttcctactgctacttcacttcaactgacttggtggcatcttggaatgagagtaaggagaactgcttccacatgggtgctcatctggtggtgatccacagccaggaagaacaggatttcatcactgggatcctggacactggtactgcttattttataggactttcaaatccaggtgatcaacaatggcaatggattgatcagacaccgtacgatgataataccacattctggcacaaaggtgagcctagcagtgacaatgaacagtgtgttataataaatcatcgtcagagtactggatggggctggagtgatatcccttgcagtgataaacagaactcaatttgccatgtgaaaaaaatatacttatga

SEQ ID NO: 6 - protein, 237 aa, Mus sp.
MFSENIYVNTNFKNKVDSSDIDTDSWPAPQRKNTSQKSCHKFSKVLFTSLIIYFLLLTILFSGALITLFTKYSQLLEEKMIIKELNYTELECTKWASLLEDKVWSCCPKDWKPFGSYCYFTSTDLVASWNESKENCFHMGAHLVVIHSQEEQDFITGILDTGTAYFIGLSNPGDQQWQWIDQTPYDDNTTFWHKGEPSSDNEQCVIINHRQSTGWGWSDIPCSDKQNSICHVKKIYL

SEQ ID NO: 7 - DNA, 29 bp, Artificial Sequence
  Oligonucleotide
agttgactcctcagacatcgacacagact

SEQ ID NO: 8 - DNA, 31 bp, Artificial Sequence
  Oligonucleotide
tggcaaattgagttctgtttatcactgcaag

SEQ ID NO: 9 - DNA, 612 bp, Mus sp.
  CDS (1)..(612)
atgttttcagaaaacatttatgttaacacgaacttcaaaaataaagttgactcctcagacatcgacacagactcttggccagctccccaaaggaagaacacgtctcagaaaagttgtcacaaattctctaaggtcctctttacctcactcataatctatttcctgctgttgacaatcttattctccggtgctctgatcaacaaagtctggagctgttgcccaaaggattggaagccgtttggttcctactgctacttcacttcaactgacttggtggcatcttggaatgagagtaaggagaactgcttccacatgggtgctcatctggtggtgatccacagccaggaagaacaggatttcatcactgggatcctggacactggtactgcttattttataggactttcaaatccaggtgatcaacaatggcaatggattgatcagacaccgtacgatgataataccacattctggcacaaaggtgagcctagcagtgacaatgaacagtgtgttataataaatcatcgtcagagtactggatggggctggagtgatatcccttgcagtgataaacagaactcaatttgccatgtgaaaaaaatatacttatga

SEQ ID NO: 10 - protein, 203 aa, Mus sp.
MFSENIYVNTNFKNKVDSSDIDTDSWPAPQRKNTSQKSCHKFSKVLFTSLIIYFLLLTILFSGALINKVWSCCPKDWKPFGSYCYFTSTDLVASWNESKENCFHMGAHLVVIHSQEEQDFITGILDTGTAYFIGLSNPGDQQWQWIDQTPYDDNTTFWHKGEPSSDNEQCVIINHRQSTGWGWSDIPCSDKQNSICHVKKIYL

SEQ ID NO: 11 - DNA, 711 bp, Mus sp.
  CDS (1)..(711)
atggcttcagaaatcacttatgcagaagtgaggatcacgaatgaatccgactccttggacacctactcaaaatgtcctgcagctcccagagagaaacctatccgtgatctaagaaagcctggttccccctcactgcttcttacatccctgatgctacttctcctgctgctggcaatcacattcttagttgcttttatcatttattttcaaaagtactctcaacttcttgaagaaaaagaagctgcaaaaaatataatgtacaaggaattgaactgcataaaaaatggttcactcatggaagacaaagtctggagctgttgcccaaaggattggaaaccatttgtttcccactgctacttcattttgaatgactcgaaggcatcttggaatgagagtgaggagaagtgctcccacatgggtgctcatctggtggtgatccacagccaggcagagcaggatttcatcaccagcaacctgaacacaagtgctggttattttataggacttttggatgctggtcaaagacaatggcgatggattgatcagacaccatacaataagagtgctacgttctggcacaaaggtgagcccaaccaagattgggaacgatgtgttataataaatcataaaacaactggatggggctggaatgatatcccttgcaaagatgaacacaattcagtttgtcaggtgaagaaaatatacttatga

SEQ ID NO: 12 - protein, 236 aa, Mus sp.
MASEITYAEVRITNESDSLDTYSKCPAAPREKPIRDLRKPGSPSLLLTSLMLLLLLLAITFLVAFIIYFQKYSQLLEEKEAAKNIMYKELNCIKNGSLMEDKVWSCCPKDWKPFVSHCYFILNDSKASWNESEEKCSHMGAHLVVIHSQAEQDFITSNLNTSAGYFIGLLDAGQRQWRWIDQTPYNKSATFWHKGEPNQDWERCVIINHKTTGWGWNDIPCKDEHNSVCQVKKIYL

SEQ ID NO: 13 - DNA, 30 bp, Artificial Sequence
  Oligonucleotide
agaagtgaggatcacgaatgaatccgactc

SEQ ID NO: 14 - DNA, 36 bp, Artificial Sequence
  Oligonucleotide
ttcttcacctgacaaactgaattgtgttcatctttg

SEQ ID NO: 15 - DNA, 443 bp, Mus sp.
  CDS (1)..(348)
atggcttcagaaatcacttatgcagaagtgaggatcacgaatgaatccgactccttggacacctactcaaaatgtcctgcagctcccagagagaaacctatccgtgatctaagaaagcctggttccccctcactgcttcttacatccctgatgctacttctcctgctgctggcaatcacattcttagttgcttttatcatttattttcaaaagtactctcaacttcttgaagaaaaagaagctgcaaaaaatataatgtacaaggaattgaactgcataaaaaatggttcactcatggaaggttctggcacaaaggtgagcccaaccaagattgggaacgatgtgttataataaatcataaaacaactggatggggctggaatgatatcccttgcaaagatgaacacaattcagtttgtcaggtgaagaaaatatacttatga

SEQ ID NO: 16 - protein, 116 aa, Mus sp.
MASEITYAEVRITNESDSLDTYSKCPAAPREKPIRDLRKPGSPSLLLTSLMLLLLLLAITFLVAFIIYFQKYSQLLEEKEAAKNIMYKELNCIKNGSLMEGSGTKVSPTKIGNDVL

SEQ ID NO: 17 - DNA, 627 bp, Mus sp.
  CDS (1)..(627)
atgatgcaggaaagaccagcccaagggcaggtagtctgctggtccctgagactctggatggctgctctgatttccatcttactcctcagcacctgtttcattgcgagttgtgtagtgacttaccagcttatgatgaacaagcccaatagaagactatctgaactccacacataccattccaatctcatctgctttagtgaaggaactacggtatcagaaaaggtctggagctgttgcccaaaggattggaagccatttggttcctactgctacttcacttcaactgactctcgggcatcccagaataagagtgaggagaagtgctctctcaggggtgctcatctggtggtgatccacagccaggaagagcaggatttcatcaccagaatgctagacactgctgctggttattttattggactttcagatgttgggaatagtcaatggcgatggattgatcagacaccatacaatgatagagccacattctggcacaaaggtgagcccaacaatgactatgaaaaatgtgttatattaaattatcggaaaactatgtggggctggaatgatattgactgcagtgatgaagagaactcagtttgtcagatgaagaaaatatacttatga

SEQ ID NO: 18 - protein, 208 aa, Mus sp.
MMQERPAQGQVVCWSLRLWMAALISILLLSTCFIASCVVTYQLMMNKPNRRLSELHTYHSNLICFSEGTTVSEKVWSCCPKDWKPFGSYCYFTSTDSRASQNKSEEKCSLRGAHLVVIHSQEEQDFITRMLDTAAGYFIGLSDVGNSQWRWIDQTPYNDRATFWHKGEPNNDYEKCVILNYRKTMWGWNDIDCSDEENSVCQMKKIYL

SEQ ID NO: 19 - DNA, 24 bp, Artificial Sequence
  Oligonucleotide
tgagactctggatggctgctctga

SEQ ID NO: 20 - DNA, 24 bp, Artificial Sequence
  Oligonucleotide
ttcttcatctgacaaactgagttc

SEQ ID NO: 21 - DNA, 472 bp, Mus sp.
  CDS (1)..(285)
atgatgcaggaaagaccagcccaagggcaggtagtctgctggtccctgagactctggatggctgctctgatttccatcttactcctcagcacctgtttcattgcgagttgtgtagtgacttaccagcttatgatgaacaagcccaatagaagactatctgaactccacacataccattccaatctcatctgctttagtgaaggaactacggtatcaggatttcatcaccagaatgctagacactgctgctggttattttattggactttcagatgttgggaatagtcaatggcgatggattgatcagacaccatacaatgatagagccacattctggcacaaaggtgagcccaacaatgactatgaaaaatgtgttatattaaattatcggaaaactatgtggggctggaatgatattgactgcagtgatgaagagaactcagtttgtcagatgaagaaaatatacttatga

SEQ ID NO: 22 - protein, 94 aa, Mus sp.
MMQERPAQGQVVCWSLRLWMAALISILLLSTCFIASCVVTYQLMMNKPNRRLSELHTYHSNLICFSEGTTVSGFHHQNARHCCWLFYWTFRCWE

SEQ ID NO: 23 - DNA, 648 bp, Homo sapiens
  CDS (1)..(648)
atggggctagaaaaacctcaaagtaaactggaaggaggcatgcatccccagctgataccttcggttattgctgtagttttcatcttacttctcagtgtctgttttattgcaagttgtttggtgactcatcacaacttttcacgctgtaagagaggcacaggagtgcacaagttagagcaccatgcaaagctcaaatgcatcaaagagaaatcagaactgaaaagtgctgaagggagcacctggaactgttgtcctattgactggagagccttccagtccaactgctattttcctcttactgacaacaagacgtgggctgagagtgaaaggaactgttcagggatgggggcccatctgatgaccatcagcacggaagctgagcagaactttattattcagtttctggatagacggctttcctatttccttggacttagagatgagaatgccaaaggtcagtggcgttgggtggaccagacgccatttaacccacgcagagtattctggcataagaatgaacccgacaactctcagggagaaaactgtgttgttcttgtttataaccaagataaatgggcctggaatgatgttccttgtaactttgaagcaagtaggatttgtaaaatacctggaacaacattgaactag

SEQ ID NO: 24 - protein, 215 aa, Homo sapiens
MGLEKPQSKLEGGMHPQLIPSVIAVVFILLLSVCFIASCLVTHHNFSRCKRGTGVHKLEHHAKLKCIKEKSELKSAEGSTWNCCPIDWRAFQSNCYFPLTDNKTWAESERNCSGMGAHLMTISTEAEQNFIIQFLDRRLSYFLGLRDENAKGQWRWVDQTPFNPRRVFWHKNEPDNSQGENCVVLVYNQDKWAWNDVPCNFEASRICKIPGTTLN

SEQ ID NO: 25 - DNA, 24 bp, Artificial Sequence
  Oligonucleotide
tctgttttattgcaagttgtttgg

SEQ ID NO: 26 - DNA, 22 bp, Artificial Sequence
  Oligonucleotide
ttccaggcccatttatcttggt